(19) United States
(12) Patent Application Publication
(10) Pub. No.: US 2026/0316036 A1
(43) Pub. Date: Oct. 15, 2026

(54) NUCLEAR HEAT INTEGRATION WITH STEAM CRACKING PROCESS

(71) Applicant: NOVA CHEMICALS (INTERNATIONAL) S.A., Fribourg (CH)

(72) Inventors: Vasily SIMANZHENKOV, Calgary (CA); Shahin GOODARZNIA, Calgary (CA); Sina MIRZAEIFARD, Calgary (CA); Mohanned MOHAMEDALI, Calgary (CA); Jeffrey KLUTHE, Calgary (CA); Joanne LAKE, Calgary (CA)

(73) Assignee: NOVA CHEMICALS (INTERNATIONAL) S.A., Fribourg (CH)

(21) Appl. No.: 19/484,802

(22) PCT Filed: May 15, 2024
(86) PCT No.: PCT/IB2024/054738

§ 371 Date: Nov. 14, 2025

Related U.S. Application Data
(60) Provisional application No. 63/503,003, filed on May 18, 2023.

(51) Int. Cl. B01J 19/00; B01J 12/00; C07C 5/327; C10G 9/36; G21C 1/32

(52) U.S. Cl. CPC B01J 19/0013; B01J 12/005; C07C 5/327; C10G 9/36; G21C 1/32; B01J 2219/00076; B01J 2219/00103; C10G 2400/20

(57) ABSTRACT

A system and methods for integrating a small modular nuclear reactor into a steam cracking process are provided. An exemplary system provides a steam cracking system that includes a small modular reactor (SMR) to provide a heat exchange stream and a steam heat exchanger to use the heat exchange stream to generate a steam stream from a water stream, wherein the steam stream is provided to a steam-cracking furnace. A feed heat exchanger uses the heat exchange stream to generate a hot feed stream, wherein the hot feed stream is provided to the steam-cracking furnace. The steam-cracking furnace to generate a product stream including an olefin.

### TECHNICAL FIELD

This disclosure relates to methods of heating a steam cracking process with heat from a nuclear reactor module.

### BACKGROUND ART

Ethane steam cracking process is an energy intensive process with a greenhouse gas (GHG) emission intensity of about 0.85 kg CO₂ eq/kg ethylene (gate-to-gate). The source of CO₂ emissions in the ethane steam crackers is attributed to fossil fuel combustion to generate heat and steam in the process. These are termed scope 1 emissions, as they are caused directly from the combustion of hydrocarbons. The main CO₂ eq emissions in the process, from the steam cracking furnaces and compressors in the downstream separation train. One technique for lowering the emission from the steam ethane crackers is to at least partially transition from steam crackers based on the combustion of fossil fuels to steam crackers based on electricity from renewable or low carbon sources (termed e-crackers). Small modular nuclear reactors (SMRs) are being studied as low carbon sources of energy, as they have a small footprint. So far, there has been no studies on direct heat integration between SMR reactors and steam cracking processes.

### SUMMARY OF INVENTION

An embodiment described herein provides a steam cracking system. The steam cracking system includes a small modular reactor (SMR) to provide a heat exchange stream and a steam heat exchanger to use the heat exchange stream to generate a steam stream from a water stream, wherein the steam stream is provided to a steam-cracking furnace. A feed heat exchanger uses the heat exchange stream to generate a hot feed stream, wherein the hot feed stream is provided to the steam-cracking furnace. The steam-cracking furnace to generate a product stream including an olefin.

Another embodiment described in examples herein provides a method for generating heat for a steam cracking process. The method includes heating a coolant stream in a small modular nuclear reactor (SMR) to create a high-temperature coolant stream. A high-pressure steam stream is generated from the high-temperature coolant stream, creating a first portion of a low-temperature coolant stream. The high-pressure steam stream is provided to a steam-cracking furnace. A hot feed stream is generated from the high-temperature coolant stream, creating a second portion of the low-temperature coolant stream. The hot feed stream is provided to the steam-cracking furnace. A product stream including an olefin is generated from the steam-cracking furnace.

Another embodiment described in examples herein provides a steam cracking process. The steam cracking process includes heating a coolant stream in a small modular nuclear reactor (SMR) to create a high-temperature coolant stream, generating a high-pressure steam stream from the high-temperature coolant stream, creating a first portion of a low-temperature coolant stream, providing the high-pressure steam stream to a steam-cracking furnace. A hot feed stream is generated from the high-temperature coolant stream, creating a second portion of the low-temperature coolant stream, wherein the hot feed stream includes a C₂ to C₆ alkane. The hot feed stream is provided to the steam-cracking furnace and a product stream including a C₂ to C₆ olefin is generated from the steam-cracking furnace.

### BRIEF DESCRIPTION OF DRAWINGS

FIG. 1 is a drawing of a steam cracking system that integrates a SMR to provide heat for a steam cracking process.

FIG. 2 is a drawing of a steam cracking system that uses a water splitter uses electricity generated from the system in an electrolysis process.

FIG. 3 is a drawing of a steam cracking system that integrates a liquid cooled SMR (LC-SMR) to provide heat for the steam cracking process.

FIG. 4 is a schematic diagram of the integration of heat from an SMR with a steam cracking process, focusing on the heat exchangers as described with respect to FIG. 1.

FIG. 5 is a schematic diagram of an integration of heat from an SMR with a steam cracking process, focusing on the heat exchangers as described with respect to FIG. 3.

FIG. 6 is a schematic diagram of an integration of the heat energy from an SMR to drive an electric turbine to generate electric power, wherein the steam effluent stream from the electric turbine is used to drive a steam-powered compressor.

FIG. 7 is a schematic diagram of an integration of the heat energy from an SMR to directly drive a steam-powered compressor.

FIG. 8 is a process flow diagram of a method for using heat from a nuclear reactor to power a steam cracking process.

FIG. 9 is a simplified process flow diagram of a simulation used to compare a combustion heated cracking process with a nuclear heated cracking process.

FIG. 10 is a schematic diagram of a startup procedure of a hydrocracking process using waste heat from a nuclear-powered steam cracking process.

FIG. 11 is a schematic diagram of startup procedure of a hydrocracking process using waste heat from a nuclear-powered steam cracking process, supplemented with heat from a furnace.

FIG. 12 is a schematic drawing of a heat integration between a fossil-based natural gas furnace and a water splitting process based on a thermochemical reaction.

FIG. 13 is a schematic diagram of a thermochemical water splitting process using an iron catalysis cycle.

FIG. 14 is a schematic diagram of a hybrid thermochemical process that uses both heat energy and electrical energy to split water.

FIG. 15 is a schematic drawing of a heat integration between an SMR heated process and a water splitting process based on a thermochemical or hybrid thermochemical reaction.

FIG. 16 is a block flow diagram of an Aspen simulation used to determine the energy gains from the integration of the thermochemical reaction with the heat energy provided by the SMR.

### DESCRIPTION OF EMBODIMENTS

The direct integration of electricity generated from small modular nuclear reactors (SMR) with chemical plants has been studied. These tests have focused on the electrification of the process, for example, to provide energy to electrically heated crackers, electric compressors, electric heaters for steam generation, and the like. For lower temperature applications, such as less than about 100° C. and medium temperature applications, such as between about 100° C. and about 500° C., the electrification of furnaces is not complicated, and some commercial electric heaters already exist. However, in the high temperature applications, such as between about 500° C. and about 1000° C., electric heaters become less efficient. Thus, these systems are not very efficient at providing heat to steam cracking furnaces.

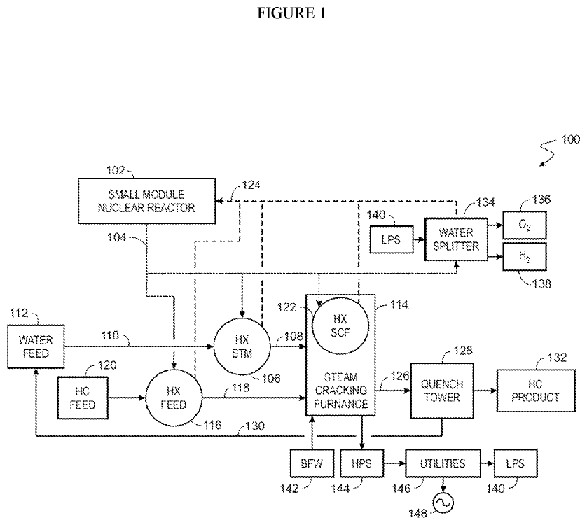

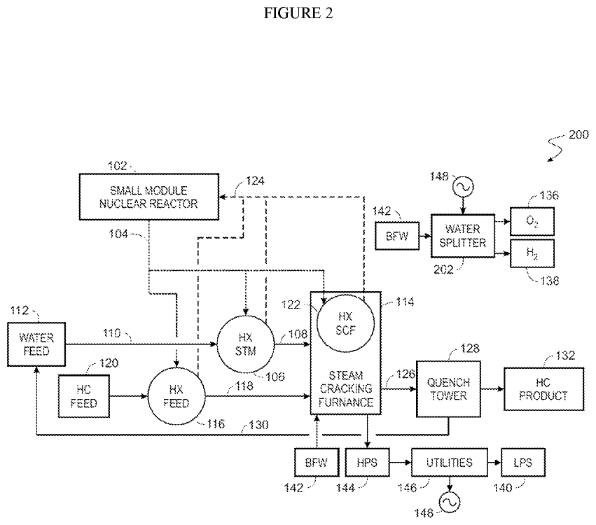

Embodiments described herein provide direct integration of heat generated in an SMR with a steam cracking process, as described further with respect to FIGS. 1 and 2. In these examples, heat from the SMR is used to generate steam for the process, heat the steam-cracking furnace directly, or both. Furthermore, the waste heat from the steam cracking process can then be used for heat integration with other processes in the plant, such as gasification, hydrocracking, or generation of low-pressure steam.

The integration of the SMR into the process provides a number of benefits. This includes a reduction in the amount of CO₂ generated in the process. Further, the methane and hydrogen generated in the steam cracking process can be provided as a product to other users, rather than being combusted inside the steam-cracking furnace. This may also eliminate the need for startup heaters in other processes, lowering the total costs. In addition, the cost of the steam-cracking furnace can be decreased by using a tube and shell heat exchanger design rather than a direct-fired heater jacket and tubing coil design.

The direct integration of the heat provided from the SMRs reduces the number of SMRs needed to satisfy the heat demand in the steam cracking process due direct heat integration, over the number that would be required if the SMRs were used to provide electric power that would be used for heating. The direct heating eliminates the inefficiencies of converting the heat from the SMRs to electricity then converting the electricity to heat. This is because the inventive concept has higher energy efficiency. Thus, this lowers the total costs by decreasing the number of SMRs needed to provide heat to the steam cracking process.

Further, other systems and methods may be integrated into the process to further utilize the heat provided by the SMR. For example, a water splitting reaction may be integrated with the heat from the SMR, or utilizing steam or power generated by the SMR. The water splitting reaction generates hydrogen, which can be used as product from the process, and oxygen, which can be used for other processes, such as an oxidative dehydrogenation (ODH) reaction. As a water splitting unit would require clean water, this can be provided by other operations such as the ODH reaction or from a supply of boiler feed water (BFW).

FIG. 1 is a drawing of a steam cracking system 100 that integrates an SMR 102 to provide heat for a steam cracking process. In the steam cracking system 100, the SMR 102 provides a hot heat exchange stream 104 that includes the reactor coolant. In this embodiment, the reactor coolant is a gas, such as a high-pressure helium stream or a CO₂ stream. The gaseous reactor coolant may be selected to not become radioactive when exposed to the high radioactivity of the reactor core. The hot heat exchange stream 104 can be directly used from the SMR 102, as the gaseous coolant will not become radioactive during flow through the core of the SMR 102.

The hot heat exchange stream 104 is provided to a steam heat exchanger 106 to generate a steam stream 108 from a water stream 110 provided from a water feed 112. The steam stream 108 is provided to a steam-cracking furnace 114. An additional steam driven heat exchanger, or electric pre-heater, may be used in place of, or in addition to, the steam heat exchanger 106. For example, the steam heat exchanger 106 can be powered by high-pressure steam from a plant steam header during periods when the SMR 102 is being serviced.

The hot heat exchange stream 104 is provided to a feed heat exchanger 116 to generate a hot feed stream 118 from a hydrocarbon feed 120. The hydrocarbon feed 120 can include a C₂ to C₆ olefin, among other components. The hot feed stream 118 is provided to the steam-cracking furnace 114. An additional steam driven heat exchanger, or electric preheater, may be used in place of, or in addition to, the feed heat exchanger 116. For example, the feed heat exchanger 116 can be powered by high-pressure steam from a plant steam header during periods when the SMR 102 is being serviced.

In this example, the hot heat exchange stream 104 is also provided to an internal heat exchanger 122 in the steam-cracking furnace 114 to provide heat to the process. The cooled heat exchange stream 124 from the heat exchangers 106, 116, and 122 is recycled to the SMR 102 to continue the process.

An effluent stream 126 from the steam-cracking furnace 114 is sent to a quench tower 128. In the quench tower 128 the cracking process is stopped, and steam is condensed to form a water stream 130 that is recycled and combined with the water feed 112.

The effluent from the quench tower 128 includes the hydrocarbon product 132, for example, a blend of hydrocarbons including an olefin product, such as an C₂ to C₆ olefin, a C₂ to C₆ alpha-olefin or other unsaturated hydrocarbons. The hydrocarbon product 132 also includes methane and hydrogen byproducts from the cracking process. The hydrocarbons and hydrogen in the blend may be isolated and provided as products, for example, to external purchasers.

As described herein, hot heat exchange stream 104 from the SMR 102 can also be provided to a water splitter 134 to provide the energy for a water splitting reaction. The water splitting reaction forms oxygen 136 and hydrogen 138, which may be sold as products, or used in other processes, such as oxidative dehydrogenation. In this embodiment, the water splitter 134 is fed low-pressure steam (LPS) 140.

The LPS 140, or saturated steam, can be generated by a number of process units. For example, BFW 142 can be fed to heat exchangers in the steam-cracking furnace 114 to generate high-pressure steam (HPS) 144. The HPS 144 can be used in utilities 146, for example, to power a steam turbine to generate electricity 148. After the removal of energy in the generation of the electricity 148, the resulting LPS 140 can be used in the water splitter 134.

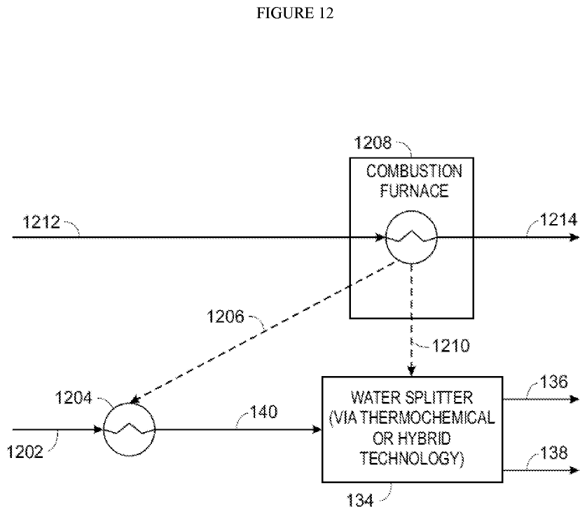

Although this integrates the generation of the LPS 140 with the heating from the SMR 102, other techniques can be used to generate the LPS 140. For example, if a steam cracking furnace is heated by the hydrogen from the water splitting process or by natural gas, the hot flue gas from the steam cracking furnace can be used to heat cold water in a heat exchanger prior to the passing the flue gas into a carbon capture unit. The cold water would be flashed to saturated steam, forming the LPS 140. This is discussed further with respect to FIG. 12.

FIG. 2 is a drawing of a steam cracking system 200 that uses a water splitter 202 uses electricity 148 generated from the system in an electrolysis process. Like numbered items are as described with respect to FIG. 1. In this embodiment, BFW 142 is fed to a water splitter 202 that uses electrolysis to split the water into the oxygen 136 and hydrogen 138. The electricity 148 for the water splitter 202 can be generated in the process from HPS 144, as described with respect to FIG. 1.

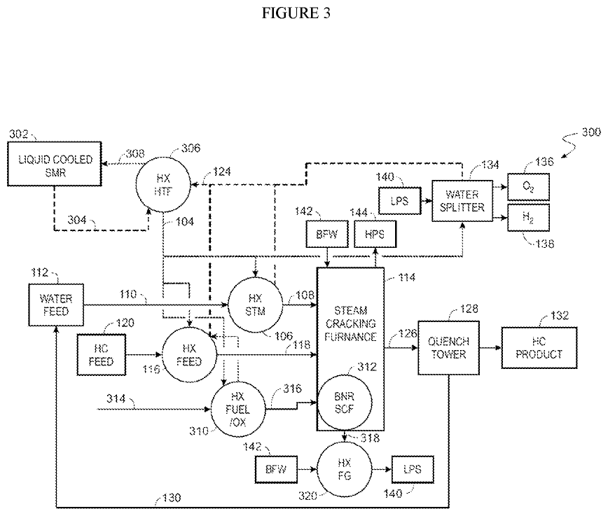

FIG. 3 is a drawing of a steam cracking system 300 that integrates a liquid cooled SMR (LC-SMR) 302 to provide heat for the steam cracking process. Like numbered items are as described with respect to FIG. 1. In this embodiment, the nuclear coolant for the LC-SMR 302 is a liquid, such as a molten salt or a liquid metal. A hot nuclear coolant stream 304 exits the LC-SMR 302 and exchanges heat with the hot heat exchange stream 104 in a high-temperature fluid (HTF) heat exchanger 306. The cooled nuclear coolant stream 308 is recycled to the LC-SMR 302. In this embodiment, the hot heat exchange stream 104 may include superheated steam, helium, CO₂, or other heat transfer fluids. The use of the HTF heat exchanger 306 isolates the radioactivity of the LC-SMR 302, as the nuclear coolant may become radioactive over time due to exposure to the radioactivity of the core.

As for the steam cracking system 100 of FIG. 1, the cooled heat exchange stream 124 is recycled for reheating, for example, in the HTF heat exchanger 306. In both embodiments, the cooled heat exchange stream 124 retains enough energy to be used for other processes, for example, heating a hydrocracking feed stream for startup, generating a low-pressure steam stream, and the like.

Rather than providing heat directly to the steam-cracking furnace 114, the hot heat exchange stream 104 may be coupled to a fuel/oxidizer (FO) heat exchanger 310 that is used to heat at least one of the fuel or the oxidizer stream provided to a burner 312 in the steam-cracking furnace 114. For example, a fuel stream 314 including methane can be provided to the FO heat exchanger 310. Further, the fuel stream 314 can include hydrogen 138 formed in the water splitting reaction, either alone or in a mixture with the methane. The hot fuel stream 316 is then provided to the burner 312.

The oxidizer may be air, for example, from a compressor (not shown), and may be passed through an isolated section of the FO heat exchanger 310 or through a separate heat exchanger. The oxidizer can be the oxygen 136 formed in the water splitting reaction, either alone or in mixtures with air.

A flue gas 318 exits the burner 312 in the steam-cracking furnace 114. Heat from the flue gas 318 may be recovered for use in other portions of the process, for example, to generate a LPS 140 in a flue-gas heat exchanger 320. After passing through the flue-gas heat exchanger 320, the CO₂ from the flue gas 318 may be recovered for carbon sequestration. The use of oxygen 136 as the oxidizer may make this process easier.

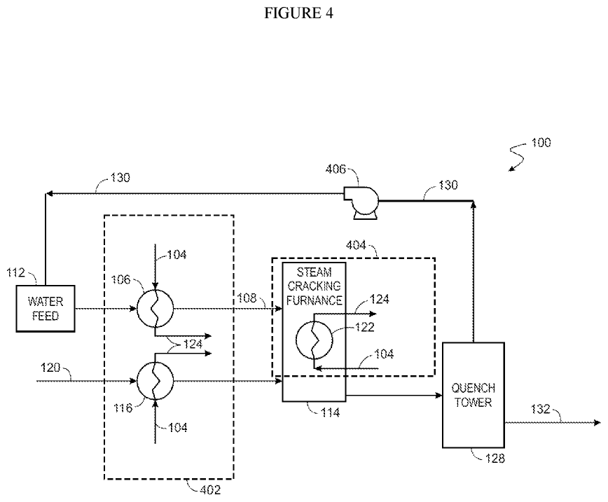

FIG. 4 is a schematic diagram of the integration of heat from an SMR with a steam cracking system 100, focusing on the heat exchangers as described with respect to FIG. 1. Like numbered items are as discussed with respect to FIG. 1. As described above, two integrations are shown in the steam cracking system 100, the heating 402 of the feedstocks and the direct heating 404 of the steam-cracking furnace 114. This allows the design of the steam-cracking furnace 114 to be changed from a fired heater furnace to lower cost system, such as a tube-and-shell heat exchanger, an adiabatic tube reactor, and the like. Accordingly, the use of the SMR modules will lower the CO₂ released from the steam cracking plant, since fuel gas is not being combusted, with the cost of the SMR modules partially being offset by the lower costs of the steam-cracking furnace 114. Further cost reductions are gained by selling the fuel gas produced as a byproduct of the steam cracking system 100.

Further, as described above, the water feed 112 and the hydrocarbon feed 120 are both heated by the hot heat exchange stream 104 from the SMR. For example, these feeds 112 and 120 may be heated to a temperature of about 800° C., about 850° C., or at least to the temperature limits of the hot heat exchange stream 104 from an SMR. Generally, the temperature of the hot heat exchange stream 104 from the SMR is limited to about 750° C. Preheating the feedstocks to increase the temperature of the materials going to the steam-cracking furnace 114, may allow it to be raised to a higher temperature more easily. A secondary integration may use electricity generated by an SMR to power electric heaters to boost the temperature from about 750° C. to between about 900° C. and 1000° C., increasing the efficiency of the steam cracking system 300. As shown in the steam cracking system 300 of FIG. 3, the pressure of the water stream 130 from the quench tower 128 is boosted by a pump 406, which helps to overcome the pressure of the steam generated in the steam heat exchanger 106.

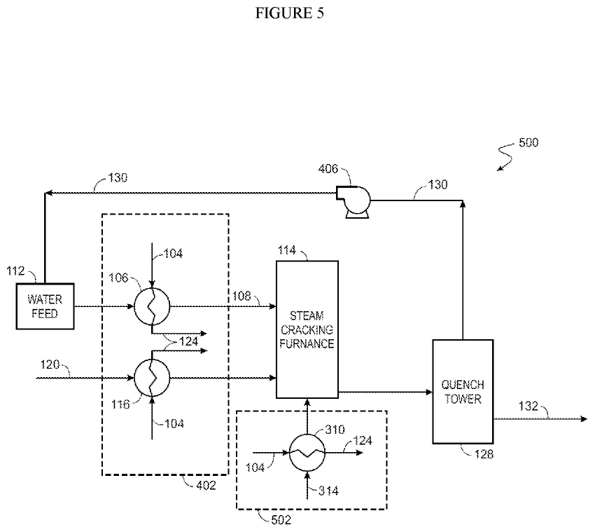

FIG. 5 is a schematic diagram of an integration of heat from an SMR with a steam cracking process 500, focusing on the heat exchangers as described with respect to FIG. 4. Like numbered items are as described with respect to FIGS. 1, 3, and 4. In this integration, the feedstocks are directly heated 402 by the hot heat exchange stream 104 from the SMR. However, heat from the SMR is used 502 in the FO heat exchanger 310 to heat at least the fuel stream 314 used in a burner in the steam-cracking furnace 114. This will reduce the amount of fuel gas needed to maintain the steam-cracking furnace 114 at the desired operating temperature of about 850° C. Further, it provides an intermediate step in the transition to using the SMR, allowing current equipment to be used in an initial integration.

In the configurations discussed with respect to FIGS. 1-4, the steam and the ethane are injected into the steam-cracking furnace 114 at an elevated temperature, for example, close to the desired steam cracking temperature of greater than about 800° C. In a normal steam cracking process, the feeds are injected into a steam-cracking furnace at a lower temperature, for example, of between about 170° C. and about 300° C., which appears to enhance the formation of an acetic acid intermediate. The acetic acid subsequently converts to COₓ in the steam cracking product stream. Thus, the integration may increase the yield of ethylene from the steam-cracking furnace as compared to conventional steam cracking furnace processes.

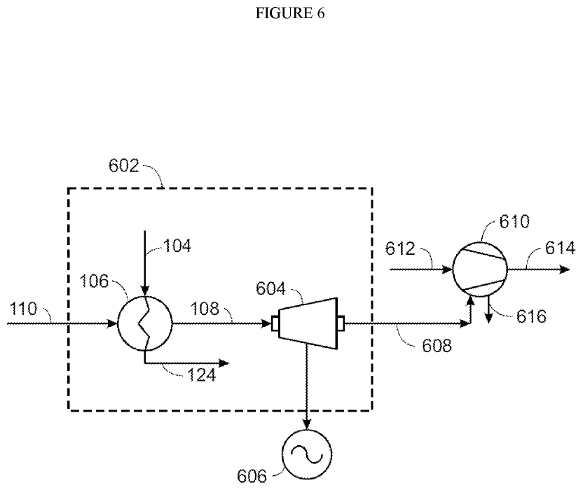

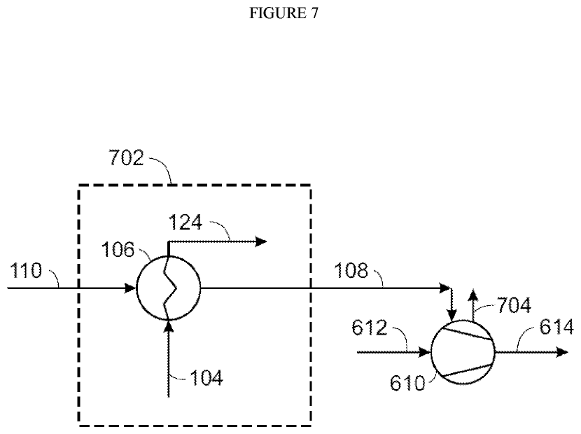

The integration of the SMR with the steam-cracking processes can be extended to other systems in the plant. For example, steam generated by heat from the SMR can be used to steam compressors, as discussed further with respect to FIGS. 6 and 7.

FIG. 6 is a schematic diagram of an integration 602 of the heat energy from an SMR to drive an electric turbine 604 to generate electric power 606, wherein the steam effluent stream 608 from the electric turbine 604 is used to drive a steam-powered compressor 610. Like numbered items are as discussed with respect to FIG. 1.

In the integration 602 shown in FIG. 6, a portion of the steam stream 108 from the steam heat exchanger 106 is used to drive an electric turbine 604. The electric turbine 604 generates electric power 606, which may be used in the process as described above, for example, to power the water splitter 202 described with respect to FIG. 2.

The steam effluent stream 608 may then be used to drive a steam-powered compressor 610. Steam-powered compressors can be used in numerous process locations, such as to compress the cracked product gas or to compress the refrigerant for distillation separation towers. Further, the electric power 606 can be used to power additional compressors.

In the steam-powered compressor 610, a low-pressure process gas or coolant stream 612 is compressed to form a high-pressure process gas or coolant stream 614. The low-pressure steam 616 that exits the steam-powered compressor 610 can be used for further heating purposes, such as HVAC, water heating, and the like. Further, the low-pressure steam 616 that exits the steam-powered compressor 610 can be provided as a feedstock to the water splitter 134.

The integration of the heat energy from the SMR is not limited to using with an electric turbine 604 but may be used to directly drive a steam power compressor as discussed with respect to FIG. 7.

FIG. 7 is a schematic diagram of an integration 702 of the heat energy from an SMR to directly drive a steam-powered compressor 610. Like numbered items are as described with respect to FIGS. 1 and 6. In this integration 702, the steam stream 108 from the steam heat exchanger 106 is used to directly drive the steam-powered compressor 610. The effluent 704 from the steam-powered compressor 610 may retain sufficient energy for use in the generation of low-pressure steam, or heating hydrocracking feed for startup, among others.

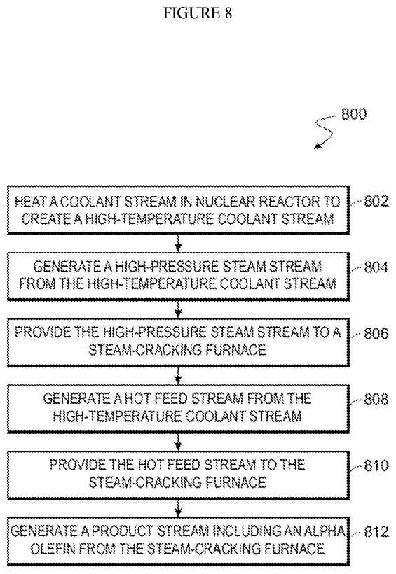

FIG. 8 is a process flow diagram of a method 800 for using heat from a nuclear reactor to power a steam cracking process. The method begins at block 802 with heating or coolant stream in a nuclear reactor to create a high temperature coolant stream. As described herein, the nuclear reactor may be a small modular nuclear reactor (SMR).

At block 804, a high-pressure steam stream is generated using the heat from the high temperature coolant stream. At block 806, the high-pressure steam stream is provided to a steam-cracking furnace as a feed stream.

At block 808, a hot feed stream is generated using the heat from the high temperature coolant stream. The heart feed stream may include an alkane, such as ethane, or combinations of an alkane with other hydrocarbons. At block 810, the hot feed stream is provided to the steam-cracking furnace.

At block 812, a product stream including an alpha olefin, such as ethylene, is generated from the steam-cracking furnace. The product stream is further separated to isolate the alpha olefin and other products, such as methane and hydrogen.

### EXAMPLES

The process of FIG. 1 was modeled using Aspen to compare the CO₂ generated using the SMR integration versus a conventional steam cracking process, with the result shown in Example 1. A further model was tested to compare the CO₂ generated from the heat integration of the SMR with an advanced recycling hydrocracking process, versus a conventional hydrocracking process, with the result shown in Example 2. Further, the use of the excess heat to power a water splitting reaction is described in Example 3.

### SMR Used for Testing:

SMR reactors refers to small to medium size nuclear reactors that generate energy from the fission of uranium fuel. The SMR used for testing was assumed to have a heat production capacity of up to 300 megawatts. Various vendors offer SMRs in this power range, such as the Xe-100 SMR available from by X-energy of Rockville, MD, USA. Generally, SMR modules have a higher heat energy output as compared to electric energy output. For example, each Xe-100 SMR module has an output of 200 MW heat energy or 80 MW electric energy. This indicates a 40% energy efficiency in converting heat energy to electric energy.

SMR modules can be selected that use different type of heat transfer fluid in the jacket of the reactor. Ethane steam cracking reactor conventionally need to be operated up to about 850° C. The SMR reactors equipped with gas heat transfer fluids (such as helium and CO₂) and liquid heat transfer fluid (such as molten salts and liquid metals) are reported to be able to generate up to 800° C., 1000° C., or 1400° C., or higher, which is suitable to provide the heat required for the ethane steam cracking furnaces.

While the SMR can supply heat suitable for operating an ethane steam-cracking furnace at desired elevated operating temperature (up to 850° C.), the design of electrically heated crackers for operation at such elevated temperature is still at pilot scale.

### Example 1: Nuclear Steam for Ethane Steam Cracking Feed Preheat and Heat of Reaction

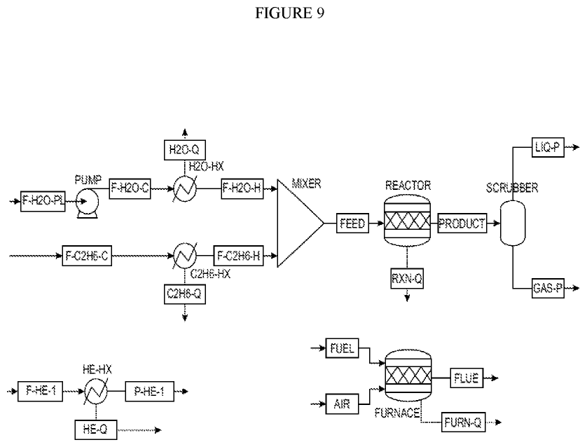

FIG. 9 is a simplified process flow diagram of a simulation used to compare a combustion heated cracking process with a nuclear heated cracking process. This corresponds to the configuration shown in FIG. 1, without the use of excess heat for water splitting. The simulation was performed using Aspen Plus model Version 11. The labels on FIG. 9 identify the streams referenced below.

In this model, the SMR was the Xe-100 SMR available from X-energy. The heat transfer fluid was helium, which is flowed through the core of the Xe-100 SMR. The operating conditions for the Xe-100 reactor are reported in Table 1. This operating condition is reflected in F—He-1 stream in FIG. 8. This stream enters a dummy heat exchanger HE-HX in which the temperature of it is dropped from about 750° C. to about 710° C. with no pressure drop to provide heat required for feed preheating, evaporation, superheating, and maintaining reaction temperature of an ethane steam cracking process. The dummy heat exchanger HP-HX models the heat exchangers 106, 116, and 122 of FIG. 1. A design specification tool was used to adjust the flow rate of helium in the F—He-1 stream such that all the required heat for the mentioned process can be satisfied in the heat exchanger He-HX.

TABLE 1
Operating Condition of Xe-100 SMR Reactor

| Parameter | Value | Unit |
|---|---|---|
| X-energy Xe100 SMR reactor | | |
| He Temperature | 750 | °C |
| He Pressure | 5899 | kPa-g |
| Thermal output per SMR module | 200 | MW |
| Electrical output per SMR module | 80 | MW |
| Efficiency of power plant | 0.4 | |

The ethane steam-cracking model, which uses the heat from the helium heat transfer fluid, is explained here. A feed water (stream FH2O-PL) enters a booster pump (PUMP) to increase the pressure of the stream. The effluent high-pressure stream (F-H2O-C) then enters a heat exchanger (H2O-HX) to transfer heat with the helium heat transfer fluid and generate a super-heated feed steam (F-H2O-H). A cold ethane feed enters a heat exchanger (C2H6-HX) to transfer heat with the helium heat transfer fluid to increase the operating temperature to close to steam cracking reaction temperature (700° C.). In this simulation, the hot feed ethane (F—C2H6-HX) is then mixed with the super-heated feed steam (F-H2O-H) to form a hot mixed feed stream (FEED) that enters the steam cracking reactor (REACTOR). The endothermic heat of reaction for ethane dehydrogenation (C₂H₆→H₂+C₂H₄) is provided by the helium heat transfer fluid in the reactor to maintain the reactor temperature at about 700° C. with a pressure drop of about 498 kPa. The reaction was assumed to have a one-pass ethane conversion to ethylene of about 65%.

The product effluent leaving the reactor (PRODUCT) then enters a separation block (SCRUBBER) to cool down the effluent to 50° C. for condensing and separating the water stream (LIQ-P) from the dry gas product stream (GAS-P). Generally, all of the water in LIQ-P is recycled back to the feed water stream (F-H2O-PL). However, to simplify the simulation, this recycle is not shown in the model. It is not expected to affect the heat balance between helium effluent from the SMR modules and the ethane steam cracking process.

The sum of heat required for this process (H2O-Q+C2H6-Q+RXN-Q) was provided by the after mentioned helium heat exchanger (HE_HX) using the mentioned design spec tool. The heat balance results are shown in shown in Table 2, which shows that 500 MW of heat is required from the SMR modules to the ethane steam cracking process. It can be noted that the flow rate of feed ethylene produced in the simulation, reflected in stream PRODUCT, is shown in Table 2 (177,798 kg/hr). The flowrates corresponds to a conventional full commercial production capacity of one ethane steam cracking process or plant.

Based on Table 1, each SMR module can provide either 200 MW of heat or 80 MW of electric power (if connected to a generator). Accordingly, either three SMR modules are needed to satisfy the steam cracking process heat demand (in the form of heat energy), or eight SMR modules are needed to satisfy the steam cracking process heat demand (in the form of electric energy). This is due to low efficiency of converting heat energy from the SMR modules to electric power energy, which is at about 40% efficiency. Therefore, direct utilization of heat energy will far more efficiently reduce the costs of the plant and process and the footprint of the SMR modules.

TABLE 2
Heat Required in Steam Cracking Furnace + Number
of SMR Heaters and/or Corresponding SMR Power Plants.

| Parameter | Value | Unit |
|---|---|---|
| Capacity of ethylene production | 177,798 | kg/hr |
| Capacity of ethylene production | 1404 | KTA* |
| Heat Energy required for ethane steam | 500 | MW |
| cracking furnace | | |
| Number of SMR modules -heat energy | 3 | |
| Number of SMR modules -electric energy | 8 | |

*Kilotons/Annum

To determine how much the CO₂ emissions are reduced by using SMR modules to replace a fuel gas fired furnace, a dummy-fired heater (FURNACE) is shown at the model in FIG. 8. The fuel gas feed (FUEL) is mixed with an air feed stream (AIR) to provide 10% additional oxygen above the molar stoichiometric ratio for full combustion of the fuel gas. The mixture is introduced to the dummy-fired heater. In the dummy-fired heater, a full combustion of flue gas is assumed (100% conversion of H₂ and CH₄). A design spec tool in the Aspen model is used to adjust the flow rate of Fuel stream such that the total heat required in the steam cracking process (H2O-Q+C2H6-Q+RXN-Q) is satisfied.

Based on this section of the model, the reduction of the mass flow rate of CO₂ emission is shown in Table 3 (47137 kg/hr). Further, the corresponding reduction of CO₂ emission intensity is calculated and shown in this table (0.27 mass ratio of CO₂/C₂H₄). To identify the percentage reduction of CO₂ emission in a whole ethane steam cracking process (feed+reactor+downstream separation), the CO₂ emission intensity of this process was collected form a public domain literature as 0.85 mass ratio of CO₂/C₂H₄. Therefore, 31% reduction in CO₂ eq intensity of a steam cracking process has been achieved by using the 3 SMR modules to provide the heat required for the feed preheat/evaporation/superheating and maintaining the ethane steam cracking reactor at 700° C. Note that steam cracker reactor typically operates at higher operating temperature of up to 850° C. The reactor temperature in this model was assumed at 700° C. for illustration purpose to indicate the benefit of the novel process configuration reported in FIG. 1. This temperature can be increased to higher temperatures because SMR gas cooled reactors can be operated up to 1000° C.

TABLE 3
CO₂ Emission Avoided Due to Using SMR Heat
Energy Module in Place of Fuel Gas.

| Parameter | Value | Unit |
|---|---|---|
| Mass flow rate of CO₂ avoided | 47137 | kg/hr |
| Mass flow rate of CO₂ avoided | 372 | KTA |
| CO₂ eq emission of whole ethane | 0.85 | CO₂ e/kg of |
| steam cracker | | ethylene |
| CO₂ intensity avoided due to using | 0.27 | mass ratio |
| nuclear energy (in place of fossil energy) | | of CO₂/C₂H₄ |
| % emission reduction in ethane | 31 | % |
| steam cracker | | |

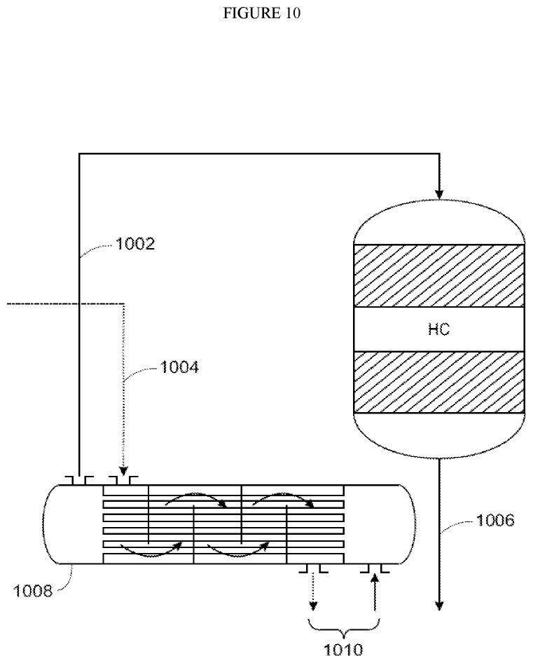

The detail mass balance is shown in Table 4. The names at the top of each column in the table identify the stream and correspond to the labels in FIG. 10.

### Example 2: Nuclear Steam for Waste AR Hydrocracking Preheat/Startup

FIG. 10 is a schematic diagram of a startup procedure of a hydrocracking process using waste heat from a nuclear-powered steam cracking process. Startup heaters/boilers are used for the standalone hydroprocessing (hydrotreating and hydrocracking) plant in order to start up. The startups of the AR plant are needed after any planned or unplanned shutdown, in particular, after each catalyst regeneration, e.g., every two months for the regeneration of catalysts. The heaters are used to initially heat the feed to the reactors, and to other equipment as needed, such as amine wash units, among others. Currently, the startup heaters/boilers are fired heaters and would be operated only for a short period during the startup of the unit after turnaround, maintenance, or any other shutdown. For this short period, these heaters will produce additional direct greenhouse gas (GHG) emissions (Scope 1 emissions caused by the combustion of natural/fuel gas), which are not present during the normal operations. Industry trend indicates that many of these heaters may become electrical. Hence, the heaters will potentially introduce indirect GHG emissions (Scope 2 emissions triggered by the generation of power/electricity). Further, regardless of the type of boilers, these heaters have associated capital costs. Therefore, waste heat derived from SMRs integrated with a steam cracking process can be used as a heating fluid in the startup heat exchanger of the Advanced Recycling (AR) plant as shown in FIG. 9.

The hydrocracking (HC) reactor inlet feed 1002 is preheated so that its temperature increases from about 25° C. to about 400° C. Generally, the feed 1004 is a mixture of 100 kta (kilotons per annum) of pyrolysis oil (py oil) and 6 kta of pure hydrogen. In normal operation, the preheating is performed by a crossflow heat exchanger located at the HC reactor outlet, on the products effluent stream 1006. In a startup mode, a heater or boiler is used to increase the feed temperature. For the simulation, the feedstock is 1-tridecene at 1 bar and 25° C. This is a proxy for py oil feed as it has a similar molecular weight of ~182 g/mol. The Aspen simulations and post-processing calculations described herein illustrate the feasibility of the heat integration. However, the absolute heat used may be different depending on the exact composition and thermophysical properties of py oil feed.

### Comparative Case

During startup, the preheating can be accomplished by either a standalone heater or a shell-and-tube heat exchanger 1008 employing a heating fluid 1010, which for the comparative case is superheated steam at 107 bar and 482.22° C., generated in a standalone boiler. Since a heat exchanger is almost 82% cheaper than a direct-fired heater for the same heat duty, the shell-and-tube heat exchanger 1008 was selected over the heater, for a comparative case. It can be noted that a boiler is used to provide the steam for the heat exchanger. The boiler is of larger duty compared to a direct-fired heater since both boiler and heat exchanger have the efficiencies less than 100%. For the sake of comparison between comparative and experimental, the proposed comparative case (boiler plus heat exchanger) is the best possible option.

### Experimental Case

The experimental process configuration is identical to the comparative case, except that the heating fluid 1010 is either hot helium or high-pressure (HP) steam, from a steam cracking plant that integrates an SMR with the steam cracking. The stream is being used for preheating after flow through the heat exchangers, for example, heat exchangers 106, 116, and 122 of the steam cracking system 100 of FIG. 1, providing a temperature increase of about 375° C. with no pressure drop, as opposed to steam generated by a standalone boiler. Please note that the superheated steam can be generated from any source in a steam cracking process, including waste steam from SMRs, startup boilers, and surplus HP steam from these steam-cracking furnace 114.

To meet the heat transfer requirement, e.g., 4.95 MW heat duty, as determined by the Aspen model, 21.98 klb/hr of superheated steam is used. Using the waste helium from FIG. 1, either from the cooled heat exchange stream 124 (710° C.) or by heat from helium-derived HP steam, this amount of steam can be readily provided by a steam cracking plant utilizing waste heat from the SMRs. This eliminates the need for a standalone boiler for the AR plant, which lowers capital expenditures, and reduces some of the greenhouse gas (GHG) emissions because of the elimination of the combustion of natural gas. Please note that in the experimental case, SMRs are already available in a steam cracking plant, hence no new reactor is required, although an increase of SMR duty is needed.

Based on available real-time data from steam cracking plants and the dynamic Aspen simulations, the savings in operating expenses and GHG emissions savings can be estimated and are shown in Table 5. During startups, a boiler may consume 6.14 MT natural gas (presumably 100% CH₄). This amount of methane gas translates into 16.83 MT CO₂ emissions. To highlight the magnitude of such emission, for example, 16.83 MT CO₂ emissions for the startup of a standalone boiler, in the AR plant, is interpreted as 2020.03 MT CO₂ emissions over the 20-year lifespan of a hydrocracking reactor, not counting emitted water vapor. The natural gas consumption and negative environmental impact can be reduced, using existing waste heat lines in steam cracking plants, by appropriate scheduling between steam cracking and hydrocracking plants operation.

TABLE 5
Summary of Savings in Capital Expenditure
(CAPEX), Operating Expenditure (OPEX), and
GHG Emissions in the Experimental Case

| | | Reductions in Experimental |
|---|---|---|
| | | Case (Compared with |
| | | Comparative Case) |
| | Units | Boiler |
| CAPEX | Million US$ | 0.3 |
| OPEX | US$ per each startup | 1.2k |
| GHG Emissions | MT CO₂ per each | 16.8 |
| | startup | |

### Hydrocracking Integration:

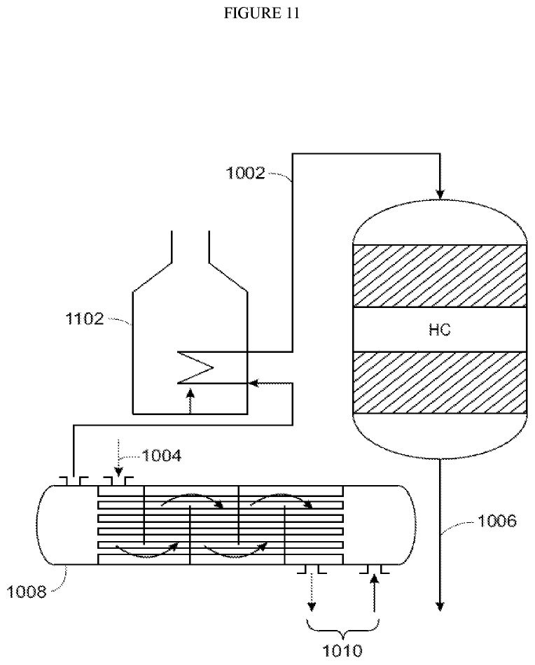

FIG. 11 is a schematic diagram of startup procedure of a hydrocracking process using waste heat from a nuclear-powered steam cracking process, supplemented with heat from a furnace 1102. Like numbered items are as described with respect to FIG. 10. In this example, the heating fluid 1010 is added in the form of high-pressure steam, for example, generated either in steam heat exchanger 106 or in a heat exchanger in the SCF 114. A secondary direct-fired heater, furnace 1102, can be added in order to allow flexibility and a proper temperature control of the inlet feed entering hydrocrackers.

### Example 3: Integration of SMR and Water Splitting Process

### Thermal Water Splitting—Comparative Example

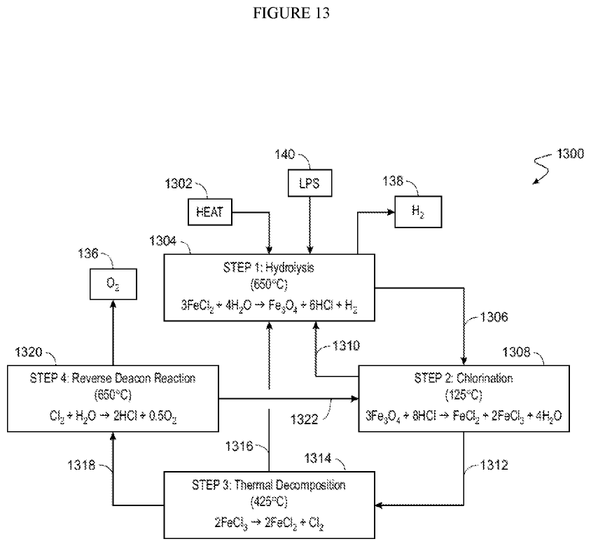

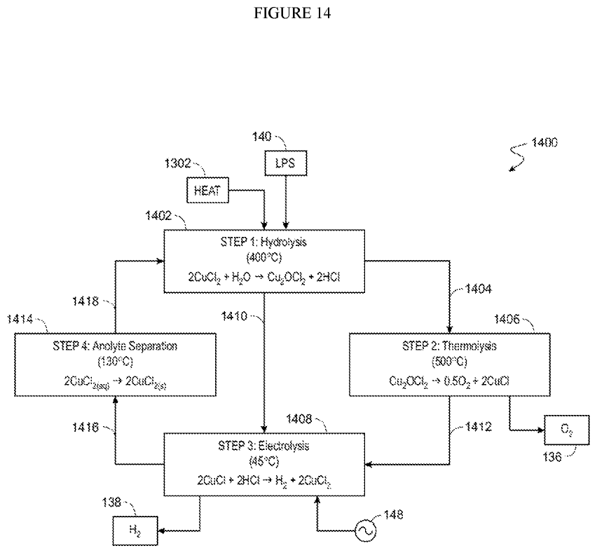

FIG. 12 is a schematic drawing of a heat integration between a fossil-based natural gas furnace and a water splitting process based on a thermochemical reaction. Like numbered items are as described with respect to FIG. 1. In this example, cold water 1202 is passed through a heat exchanger 1204 to generate a low-pressure steam (LPS) 140. The LPS 140 is fed to a water splitter 134 which uses the heat from the LPS 140 to provide energy to a water splitting reaction. The water splitting reaction may be a thermochemical reaction as described with respect to FIG. 13 or a hybrid system using both a thermochemical reaction and electrolysis, as described further with respect to FIG. 14.

In this example, heat energy 1206 is provided to the heat exchanger 1204 from a combustion furnace 1208, for example, as a portion of a flue gas from the combustion furnace. Additional heat energy 1210 can be used to directly heat the water splitter 134 from the flue gas of the combustion furnace 1208. As described with respect to FIG. 1, the water splitter forms hydrogen 138 and oxygen 136. The combustion furnace 1208 has inlet streams 1212 of fuel and oxidizer. In this example, excess flue gas 1214 not needed for heating is released to the atmosphere.

### Thermochemical Water Splitting Using Iron Catalysis.

FIG. 13 is a schematic diagram of a thermochemical water splitting process 1300 using an iron catalysis cycle. Like numbered items are as described with respect to FIG. 1. In this process, the only source of energy is provided by heat 1302, which may be from an LPS 140. Additional heat may be provided to the process from an SMR, as described with respect to FIGS. 1-3, or from a combustion furnace, as described with respect to FIG. 12. The LPS 140 also provides a source of water for the reaction. The thermochemical water splitting process 1300 is a four-step process, wherein the first step is hydrolysis 1304.

In hydrolysis 1304, FeCl₂ (iron (II) chloride) reacts with the water at a high temperature to form Fe₃O₄ (iron (II, III) oxide), releasing HCl and hydrogen 138. The reaction takes place at about 650° C. As indicated by an arrow 1306, the Fe₃O₄ and HCl are provided to step two, chlorination 1308.

The chlorination 1308 is performed at about 125° C. In the chlorination 1308, the Fe₃O₄ reacts with the HCl to form FeCl₂, FeCl₃, and water. As indicated by an arrow 1310, the FeCl₂ and water are returned to the hydrolysis 1304. As indicated by arrow 1312, the FeCl₃ is provided to step three, which is thermal decomposition 1314.

In thermal decomposition 1314, the FeCl₃ is decomposed to form FeCl₂ and chlorine. The thermal decomposition 1314 operates at about 425° C. As indicated by arrow 1316, the FeCl₂ is returned to the hydrolysis 1304. The chlorine, as indicated by arrow 1318 is provided to step four, which is a reverse Deacon reaction 1320.

In the reverse Deacon reaction 1320, chlorine is reacted with water vapor at a high temperature to form HCl and oxygen 136. The reverse deacon reaction 1320 is run at about 650° C. The oxygen 136 is removed as a product stream, while the HCl is provided to the chlorination 1308, as indicated by arrow 1322.

The techniques described herein are not limited to the thermochemical water splitting process 1300 but may use other catalytic chemistries or energy inputs. For example, as described with respect to FIG. 14, a hybrid technique may be used, wherein both thermal energy and electrical energy drive the formation of hydrogen 138 and oxygen 136.

FIG. 14 is a schematic diagram of a hybrid thermochemical process 1400 that uses both heat energy and electrical energy to split water. Like numbered items are as described with respect to FIGS. 1 and 13. In this example, a copper catalytic cycle is used. As for the thermochemical water splitting process 1300, the hybrid thermochemical process 1400 is a four-step process, wherein the first step is hydrolysis 1402. LPS 140 is provided to the hydrolysis 1402 to provide water as a reacted and heat to power the process. Further heat 1302 can be added from other process units, such as the SMR 102 described with respect to FIG. 1, or other units described with respect to FIG. 13.

In the hydrolysis 1402, CuCl₂ is reacted with water at a high temperature to form Cu₂OCl₂ and HCl. The hydrolysis 1402 is operated at a temperature of about 400° C. As indicated by arrow 1404, the Cu₂OCl₂ is passed on to step two, thermolysis 1406. The HCl is passed to step three, electrolysis 1408, as indicated by arrow 1410.

In the thermolysis 1406, the Cu₂OCl₂ is decomposed at a temperature of about 500° C., forming oxygen 136 and CuCl. The oxygen 136 is removed as a product. The CuCl is passed to the electrolysis 1408, as indicated by arrow 1412.

In the electrolysis 1408, electricity 148 he is used to drive an electrolysis reaction in which CuCl is reacted with HCl to form hydrogen 138 and CuCl₂. The electrolysis is performed at about 45° C. The hydrogen 138 is removed as a product. The CuCl₂ is passed on to step four, analyte separation 1414, as indicated by arrow 1416.

In the analyte separation 1414, CuCl₂(aq) is dried to form CuCl₂(s) at about 130° C. In some embodiments, the CuCl₂(aq) is not completely dried, but water is removed to form a more concentrated solution. The CuCl₂(s) is then returned to the hydrolysis 1402, as indicated by arrow 1418.

### Thermal Water Splitting—Experimental Example

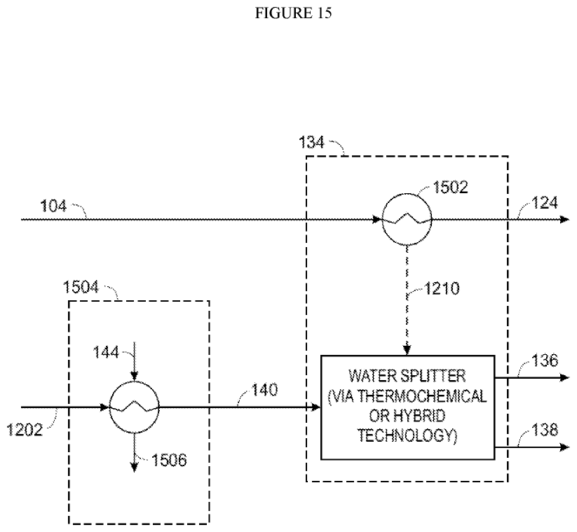

FIG. 15 is a schematic drawing of a heat integration between an SMR heated process and a water splitting process based on a thermochemical or hybrid thermochemical reaction. Like numbered items are as described with respect to FIGS. 1 and 12. In this example, cold water 1202 is passed through a heat exchanger 1504 to generate a low-pressure steam (LPS) 140. The LPS 140 is fed to a water splitter 134 which uses the heat from the LPS 140 to provide energy to a water splitting reaction. In this example, the heat exchanger 1504 is heated by a flow of high-pressure steam (HPS) 144, for example, generated in a heat exchanger in a steam cracking reactor, although other sources of heat, such as a separate heat exchanger heated by coolant flow from an SMR, or a direct coolant flow from an SMR, can be used to generate the LPS 140. A cooled water stream 1506 can be returned to a water cooler, for example, in a chilled water or boiler feed water system.

The water splitting reaction may be a thermochemical reaction as described with respect to FIG. 13 or a hybrid system using both a thermochemical reaction and electrolysis, as described further with respect to FIG. 14.

As described with respect to FIG. 1, the water splitter 134 forms hydrogen 138 and oxygen 136. In addition to the heat from the LPS 140, the additional heat 1210 can be provided from one or more heat exchangers 1502 incorporated into the water splitter 134. For example, the heat exchangers 1502 can be heated by a portion of a hot heat exchange stream 104 from the SMR 102 forming a cooled heat exchange stream 124, which can be combined with other streams and returned to the SMR 102.

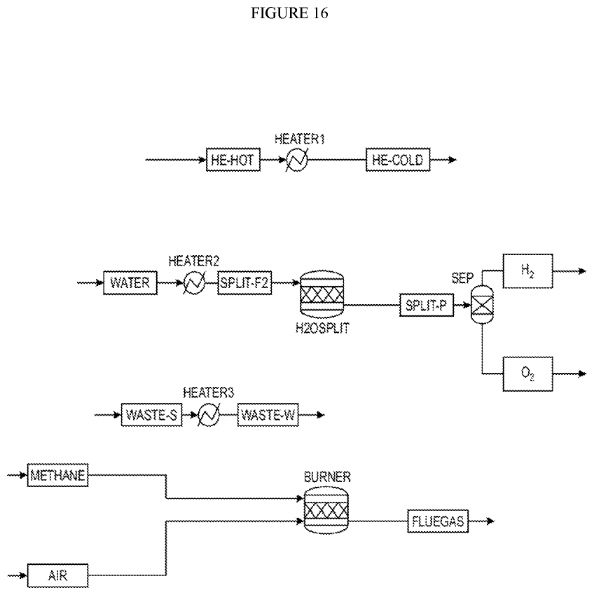

FIG. 16 is a block flow diagram of an Aspen simulation used to determine the energy gains from the integration of the thermochemical reaction with the heat energy provided by the SMR. The objective of the simulation example is to model the comparative case (FIG. 12) and experimental case (FIG. 15) to determine the CO2 intensity avoidance in generating O2 from thermochemical water splitting (t-water splitting) in an integration with an SMR. Aspen Plus V.12.1 is used for this simulation. The “Peng-Rob” equation of state property package was used for all of the streams in this model except for pure H2O streams in which the “Steam-TA” equation of state was used.

The net heat required for t-water splitting reactor (“H2OSPLIT”) was collected from references is reflected in the table 4. The hottest part of this unit was operated at 500° C. The unit was operated at inlet pressure of 131.3 kPa-a.

The Waste steam (“Waste-S”) from the boiler feed water (BFW) circulation system was assumed to be operating at 155° C. at 323 kPa-g. This operating condition is a typical waste steam specification from a conventional commercial ethane steam cracking unit.

The hot waste helium heat transfer fluid (“He-HOT”) from steam cracking nuclear reactor module specifications (temperature, pressure, and flow rate) were taken from stream “P-HE-1” stream from the first simulation example, described with respect to FIG. 9. In all heat exchangers and reactor units in this simulation a10 kPa pressure drop was assumed.

In the example shown in FIG. 16, a water feed stream (“WATER”) at 66° C. and 30 kPa-g enters a heat exchanger (“HEATER2”) to be converted to a saturated steam (“SPLIT-F2”). The heat required for water evaporation is provided by exchanging heat with heat exchanger (“HEATER3”). In “HEATER3” heat exchanger, the waste steam (“Waste-S”) is condensed to provide the heat required for “Heater2” heat exchanger. The saturated steam (“SPLIT-F2”) enters the t-water splitter reactor (“H2OSPLIT”) to be converted to H2 (“H2” stream) and O2 (“O2” stream) using the simplified bulk reaction listed below. The net heat of reaction for this reactor is provided by “HEATER1” heat exchanger. In this heat exchanger, the hot waste helium heat transfer fluid (“HE-HOT”) is cooled down from 710° C. to 570° C. which results in cool waste helium stream (“HE-COLD”).

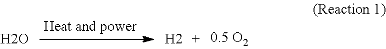

The flow rate of feed water (“WATER”) was identified using an Aspen Design Spec such that the heat of reaction “H2OSPLIT” becomes equal to “HEATER1” heat exchanger.

The description above is for the simulation of the experimental example of FIG. 15. To generate the simulation for the comparative example of FIG. 12, the following additions were made to the simulation. The heat required for “H2OSPLIT” reactor and “HEATER2” heat exchanger can alternatively be provided using a fossil-based combustion of methane in an R-Stoic reactor (“BURNER”). In the “BURNER”, the methane feed (Methane”) is mixed with the air feed (“Air”) at a 110 mol % stoichiometric O2 concentration for full methane combustion, according to RXN 2.

CH₄+2O₂→CO₂+2H₂O RXN 2

The CO₂ generated in “BURNER” reactor is assumed to be released to the environment leading to greenhouse gas (GHG) emissions. The simulations were used to determine the CO₂ intensities, or GHG emissions, for the simulated scenarios.

Table 6 summarizes the CO₂ intensities of the t-water splitter process for three heat sources. These include the heat from the earth SMR/waste-steam steams (CO₂ intensity 1), combustion of fossil-based methane (CO₂ intensity 2) and fossil-fuel based cryogenic air separation unit ASU (CO₂ intensity 3). CO₂ intensity 3, used for comparative purposes, was not modeled. The data for this was obtained from references. The results in table six shows full elimination of CO₂ emission for 02 generation via t-water splitting (via heat source 1) as compared to t-water splitting (via heat source 2 or 3).

TABLE 6
CO₂ Emission Avoided Due to Using SMR Heat Energy Module and
Waste Steam Form Steam Cracking Process in Place of Fuel Gas.

| Parameter | Value | Unit |
|---|---|---|
| CO₂ avoided (“FlueGas” Stream) | 22193 | kg/hr |
| O₂ generated (“O2” stream) | 112914 | kg/hr |
| CO₂ intensity 1 of t-water splitter process | 0 | kg CO2/ |
| (using SMR/waste-steam steams) | | kg O2 |
| CO₂ intensity 2 of t-water splitter process | 0.20 | kg CO2/ |
| (using combustion of fossil-based methane) | | kg O2 |
| CO₂ intensity 3 of t-water splitter process | 0.12 | kg CO2/ |
| (fossil-based ASU) | | kg O2 |

### EMBODIMENTS

An embodiment described herein provides a steam cracking system. The steam cracking system includes a small modular reactor (SMR) to provide a heat exchange stream and a steam heat exchanger to use the heat exchange stream to generate a steam stream from a water stream, wherein the steam stream is provided to a steam-cracking furnace. A feed heat exchanger uses the heat exchange stream to generate a hot feed stream, wherein the hot feed stream is provided to the steam-cracking furnace. The steam-cracking furnace to generate a product stream including an olefin.

In an aspect, combinable with any other aspect, the steam-cracking furnace includes an internal heat exchanger coupled to the heat exchange stream.

In an aspect, combinable with any other aspect, the steam-cracking furnace includes an internal heat exchanger coupled to a fuel gas stream, wherein the internal heat exchanger is configured to combust the fuel gas stream.

In an aspect, combinable with any other aspect, the steam-cracking furnace includes a fuel gas heat exchanger to heat the fuel gas stream using the heat exchange stream.

In an aspect, combinable with any other aspect, the internal heat exchanger is coupled to an oxidizer stream, wherein the oxidizer stream is heated in an oxidizer heat exchanger.

In an aspect, combinable with any other aspect, the heat exchange stream includes a gas stream heated in the SMR.

In an aspect, combinable with any other aspect, the gas stream includes helium.

In an aspect, combinable with any other aspect, the gas stream includes carbon dioxide.

In an aspect, combinable with any other aspect, the steam-cracking system includes a nuclear coolant heat exchanger to generate the heat exchange stream. In an aspect, combinable with any other aspect, the nuclear coolant includes a liquid metal. In an aspect, the nuclear coolant includes a molten salt. In an aspect, the nuclear coolant includes a hot gas.

In an aspect, combinable with any other aspect, the steam-cracking system includes a quench tower coupled to an effluent line from the steam-cracking furnace.

In an aspect, combinable with any other aspect, the steam-cracking system includes a water stream from the quench tower coupled to the water stream to the steam heat exchanger.

In an aspect, combinable with any other aspect, the steam-cracking system includes a low-pressure steam generator to generate steam from the heat exchange stream downstream of the steam heat exchanger.

In an aspect, combinable with any other aspect, the steam-cracking system includes a low-pressure steam generator to generate steam from the heat exchange stream downstream of the feed heat exchanger, the steam heat exchanger, or the internal heat exchanger, or any combination thereof.

In an aspect, combinable with any other aspect, the steam-cracking system includes a low-pressure steam generator to generate steam from a cold-water stream exchanging heat with a boiler feed water waste stream.

In an aspect, combinable with any other aspect, the steam-cracking system includes a power turbine coupled to the steam stream from the steam heat exchanger to generate electrical power, wherein a steam effluent from the power turbine is used to power a steam-powered compressor.

In an aspect, combinable with any other aspect, the steam-cracking system includes an electric powered compressor powered by the electrical power from the power turbine.

In an aspect, combinable with any other aspect, the steam-cracking system includes a steam-powered compressor powered by the steam stream from the steam heat exchanger.

In an aspect, combinable with any other aspect, the steam cracking system includes an electrolytic water splitter, wherein the water splitter generates oxygen and hydrogen. In an aspect, the electrolytic water splitter is powered by electricity generated in the steam cracking system. In an aspect, the electrolytic water splitter is fed a boiler feed water.

In an aspect, combinable with any other aspect, the steam cracking system includes a thermochemical water splitter, wherein the thermochemical water splitter is coupled to the heat exchange stream, wherein the thermochemical water splitter is fed a low-pressure steam wherein the water splitter generates oxygen and hydrogen.

In an aspect, the thermochemical water splitter uses an iron catalytic cycle.

In an aspect, the thermochemical water splitter is coupled to the heat exchange stream, and wherein the water splitter is fed a low-pressure steam.

In an aspect, the thermochemical water splitter uses a copper catalytic cycle, and wherein the copper catalytic cycle comprises an electrolytic reaction. In an aspect, the thermochemical water splitter is powered by electricity generated in the steam cracking system.

Another embodiment described in examples herein provides a method for generating heat for a steam cracking process. The method includes heating a coolant stream in a small modular nuclear reactor (SMR) to create a high-temperature coolant stream. A high-pressure steam stream is generated from the high-temperature coolant stream, creating a first portion of a low-temperature coolant stream. The high-pressure steam stream is provided to a steam-cracking furnace. A hot feed stream is generated from the high-temperature coolant stream, creating a second portion of the low-temperature coolant stream. The hot feed stream is provided to the steam-cracking furnace. A product stream including an olefin is generated from the steam-cracking furnace.

In an aspect, the method includes providing heat to the steam-cracking furnace from the high-temperature coolant stream, creating a third portion of the low-temperature coolant stream.

In an aspect, combinable with any other aspect, the method includes generating a high temperature fuel stream from the high-temperature coolant stream and combusting the high temperature fuel stream to heat the steam-cracking furnace.

In an aspect, combinable with any other aspect, generating the high-pressure steam stream from the high-temperature coolant stream includes exchanging heat between the high-temperature coolant stream and a water stream.

In an aspect, combinable with any other aspect, generating the high-pressure steam stream from the high-temperature coolant stream includes heating a heat exchange fluid with the high-temperature coolant stream, and exchanging heat between the heat exchange fluid and water stream to generate the steam stream, generating a low temperature heat exchange stream.

In an aspect, combinable with any other aspect, generating the hot feed stream from the high-temperature coolant stream includes exchanging heat between the high-temperature coolant stream and a cold feed stream.

In an aspect, combinable with any other aspect, generating the hot feed stream from the high-temperature coolant stream includes heating a heat exchange stream with the high-temperature coolant stream, and exchanging heat between the heat exchange stream and a cold-water stream to generate the hot feed stream.

In an aspect, combinable with any other aspect, the method includes powering a turbine with the high-pressure steam stream to generate electricity and powering a compressor with a steam effluent from the turbine.

In an aspect, combinable with any other aspect, the method includes powering a compressor with the high-pressure steam stream.

In an aspect, combinable with any other aspect, the method includes using the low-temperature coolant stream to heat a feed for a hydrocracking unit.

In an aspect, combinable with any other aspect, the method includes using the low-temperature coolant stream to generate a low-pressure steam stream.

In an aspect, the low-pressure steam is fed to a water splitter. In an aspect, the method comprises splitting water in the water splitter using a thermochemical reaction based on an iron catalytic cycle.

In an aspect, the method comprises splitting water in the water splitter using an electro-thermochemical reaction based on a copper catalytic cycle.

In an aspect, the method comprises splitting water in an electrolytic water splitter, wherein the electrolytic water splitter is fed a boiler feed water, and wherein the electrolytic water splitter is powered by electricity generated in the process.

Another embodiment described in examples herein provides a steam cracking process. The steam cracking process includes heating a coolant stream in a small modular nuclear reactor (SMR) to create a high-temperature coolant stream, generating a high-pressure steam stream from the high-temperature coolant stream, creating a first portion of a low-temperature coolant stream, providing the high-pressure steam stream to a steam-cracking furnace. A hot feed stream is generated from the high-temperature coolant stream, creating a second portion of the low-temperature coolant stream, wherein the hot feed stream includes a C₂ to C₆ alkane. The hot feed stream is provided to the steam-cracking furnace and a product stream including a C₂ to C₆ olefin is generated from the steam-cracking furnace.

In an aspect, combinable with any other aspect, the process includes heating the steam-cracking furnace with heat from the high-temperature coolant stream.

In an aspect, combinable with any other aspect, the process includes heating the steam-cracking furnace with heat from an internal combustor.

In an aspect, combinable with any other aspect, the process includes heating a fuel gas to the internal combustor with heat from the high-temperature coolant stream.

In an aspect, combinable with any other aspect, the process includes generating steam to power a steam compressor with heat from the low-temperature coolant stream.

Other implementations are also within the scope of the following claims.

TABLE 4
Modeling Results from ASPEN for FIG. 8 (column headers identify units)

| | | AIR | F-C2H6-C | F-C2H6-H | F-H2O-C | F-H2O-H | F-H2O-PL | F-HE-1 |
|---|---|---|---|---|---|---|---|---|
| | | From | | | | | | |
| | | | | C2H6-HX | PUMP | H2O-HX | | |
| | | To | | | | | | |
| | | FURNACE | C2H6-HX | MIXER | H2O-HX | MIXER | PUMP | HE-HX |
| | | Stream Class | | | | | | |
| Description | Units | CONVEN | CONVEN | CONVEN | CONVEN | CONVEN | CONVEN | CONVEN |
| Maximum | | | | | | | | |
| Relative Error | | | | | | | | |
| Cost Flow | $/sec | | | | | | | |
| MIXED | | | | | | | | |
| Substream | | | | | | | | |
| Phase | | Vapor Phase | Vapor Phase | Vapor Phase | Liquid Phase | Vapor Phase | Liquid Phase | Vapor Phase |
| Temperature | C | 25 | 105 | 700 | 50.0310474 | 172 | 50 | 750 |
| Pressure | kPag | 101 | 749.2 | 739.2 | 705 | 695 | 97 | 5899 |
| Molar Vapor | | 1 | 1 | 1 | 0 | 1 | 0 | 1 |
| Fraction | | | | | | | | |
| Molar Liquid | | 0 | 0 | 0 | 1 | 0 | 1 | 0 |
| Fraction | | | | | | | | |
| Molar Solid | | 0 | 0 | 0 | 0 | 0 | 0 | 0 |
| Fraction | | | | | | | | |
| Mass Vapor | | 1 | 1 | 1 | 0 | 1 | 0 | 1 |
| Fraction | | | | | | | | |
| Mass Liquid | | 0 | 0 | 0 | 1 | 0 | 1 | 0 |
| Fraction | | | | | | | | |
| Mass Solid | | 0 | 0 | 0 | 0 | 0 | 0 | 0 |
| Fraction | | | | | | | | |
| Molar Enthalpy | J/kmol | −16244.31394 | −79574692.68 | −22687880.6 | −285689787.5 | −237262954 | −285702340.6 | 15147294.16 |
| Mass Enthalpy | J/kg | −563.0533899 | −2646346.703 | −754511.2147 | −15858193.02 | −13170095.27 | −15858889.82 | 3784363.704 |
| Molar Entropy | J/kmol-K | −1521.997954 | −178789.1606 | −92551.18613 | −161414.8636 | −48592.00864 | −161413.1772 | −8306.636333 |
| Mass Entropy | J/kg-K | −52.75483535 | −5945.836418 | −3077.894718 | −8959.886472 | −2697.266356 | −8959.792867 | −2075.310132 |
| Molar Density | kmol/cum | 0.081706077 | 0.280368804 | 0.103721129 | 53.81392819 | 0.224931237 | 53.81563816 | 0.698650186 |
| Mass Density | kg/cum | 2.357252764 | 8.430588992 | 3.118856998 | 969.4729842 | 4.052199223 | 969.5037899 | 2.796417233 |
| Enthalpy Flow | MW | −0.100990258 | −215.523555 | −61.44884157 | −387.4564902 | −321.7793406 | −387.473515 | 9103.031671 |
| Average MW | | 28.8503972 | 30.06964 | 30.06964 | 18.01528 | 18.01528 | 18.01528 | 4.0026 |
| Mole Flows | kmol/hr | 22381.05775 | 9750.396413 | 9750.396413 | 4882.37041 | 4882.37041 | 4882.37041 | 2163483.04 |
| C2H6 | kmol/hr | 0 | 9750.396413 | 9750.396413 | 0 | 0 | 0 | 0 |
| C2H4 | kmol/hr | 0 | 0 | 0 | 0 | 0 | 0 | 0 |
| H2 | kmol/hr | 0 | 0 | 0 | 0 | 0 | 0 | 0 |
| HE | kmol/hr | 0 | 0 | 0 | 0 | 0 | 0 | 2163483.04 |
| H2O | kmol/hr | 0 | 0 | 0 | 4882.37041 | 4882.37041 | 4882.37041 | 0 |
| CH4 | kmol/hr | 0 | 0 | 0 | 0 | 0 | 0 | 0 |
| O2 | kmol/hr | 4700.022128 | 0 | 0 | 0 | 0 | 0 | 0 |
| N2 | kmol/hr | 17681.03562 | 0 | 0 | 0 | 0 | 0 | 0 |
| CO2 | kmol/hr | 0 | 0 | 0 | 0 | 0 | 0 | 0 |
| Mole Fractions | | | | | | | | |
| C2H6 | | 0 | 1 | 1 | 0 | 0 | 0 | 0 |
| C2H4 | | 0 | 0 | 0 | 0 | 0 | 0 | 0 |
| H2 | | 0 | 0 | 0 | 0 | 0 | 0 | 0 |
| HE | | 0 | 0 | 0 | 0 | 0 | 0 | 1 |
| H2O | | 0 | 0 | 0 | 1 | 1 | 1 | 0 |
| CH4 | | 0 | 0 | 0 | 0 | 0 | 0 | 0 |
| O2 | | 0.21 | 0 | 0 | 0 | 0 | 0 | 0 |
| N2 | | 0.79 | 0 | 0 | 0 | 0 | 0 | 0 |
| CO2 | | 0 | 0 | 0 | 0 | 0 | 0 | 0 |
| Mass Flows | kg/hr | 645702.4059 | 293190.91 | 293190.91 | 87957.27 | 87957.27 | 87957.27 | 8659557.214 |
| C2H6 | kg/hr | 0 | 293190.91 | 293190.91 | 0 | 0 | 0 | 0 |
| C2H4 | kg/hr | 0 | 0 | 0 | 0 | 0 | 0 | 0 |
| H2 | kg/hr | 0 | 0 | 0 | 0 | 0 | 0 | 0 |
| HE | kg/hr | 0 | 0 | 0 | 0 | 0 | 0 | 8659557.214 |
| H2O | kg/hr | 0 | 0 | 0 | 87957.27 | 87957.27 | 87957.27 | 0 |
| CH4 | kg/hr | 0 | 0 | 0 | 0 | 0 | 0 | 0 |
| O2 | kg/hr | 150395.0681 | 0 | 0 | 0 | 0 | 0 | 0 |
| N2 | kg/hr | 495307.3378 | 0 | 0 | 0 | 0 | 0 | 0 |
| CO2 | kg/hr | 0 | 0 | 0 | 0 | 0 | 0 | 0 |
| Mass Fractions | | | | | | | | |
| C2H6 | | 0 | 1 | 1 | 0 | 0 | 0 | 0 |
| C2H4 | | 0 | 0 | 0 | 0 | 0 | 0 | 0 |
| H2 | | 0 | 0 | 0 | 0 | 0 | 0 | 0 |
| HE | | 0 | 0 | 0 | 0 | 0 | 0 | 1 |
| H2O | | 0 | 0 | 0 | 1 | 1 | 1 | 0 |
| CH4 | | 0 | 0 | 0 | 0 | 0 | 0 | 0 |
| O2 | | 0.232917001 | 0 | 0 | 0 | 0 | 0 | 0 |
| N2 | | 0.767082999 | 0 | 0 | 0 | 0 | 0 | 0 |
| CO2 | | 0 | 0 | 0 | 0 | 0 | 0 | 0 |
| Volume Flow | cum/sec | 76.08932828 | 9.660288211 | 26.11274563 | 0.025201914 | 6.029460462 | 0.025201113 | 860.1837133 |
| Vapor Phase | | | | | | | | |
| Molar Enthalpy | | −16244.31394 | −79574692.68 | −22687880.6 | | −237262954 | | 15147294.16 |
| Mass Enthalpy | | −563.0533899 | −2646346.703 | −754511.2147 | | −13170095.27 | | 3784363.704 |
| Molar Entropy | | −1521.997954 | −178789.1606 | −92551.18613 | | −48592.00864 | | −8306.636333 |
| Mass Entropy | | −52.75483535 | −5945.836418 | −3077.894718 | | −2697.266356 | | −2075.310132 |
| Molar Density | | 0.081706077 | 0.280368804 | 0.103721129 | | 0.224931237 | | 0.698650186 |
| Mass Density | | 2.357252764 | 8.430588992 | 3.118856998 | | 4.052199223 | | 2.796417233 |
| Enthalpy Flow | | −0.100990258 | −215.523555 | −61.44884157 | | −321.7793406 | | 9103.031671 |
| Average MW | | 28.8503972 | 30.06964 | 30.06964 | | 18.01528 | | 4.0026 |
| Mole Flows | kmol/hr | 22381.05775 | 9750.396413 | 9750.396413 | | 4882.37041 | | 2163483.04 |
| C2H6 | kmol/hr | 0 | 9750.396413 | 9750.396413 | | 0 | | 0 |
| C2H4 | kmol/hr | 0 | 0 | 0 | | 0 | | 0 |
| H2 | kmol/hr | 0 | 0 | 0 | | 0 | | 0 |
| HE | kmol/hr | 0 | 0 | 0 | | 0 | | 2163483.04 |
| H2O | kmol/hr | 0 | 0 | 0 | | 4882.37041 | | 0 |
| CH4 | kmol/hr | 0 | 0 | 0 | | 0 | | 0 |
| O2 | kmol/hr | 4700.022128 | 0 | 0 | | 0 | | 0 |
| N2 | kmol/hr | 17681.03562 | 0 | 0 | | 0 | | 0 |
| CO2 | kmol/hr | 0 | 0 | 0 | | 0 | | 0 |
| Mole Fractions | | | | | | | | |
| C2H6 | | 0 | 1 | 1 | | 0 | | 0 |
| C2H4 | | 0 | 0 | 0 | | 0 | | 0 |
| H2 | | 0 | 0 | 0 | | 0 | | 0 |
| HE | | 0 | 0 | 0 | | 0 | | 1 |
| H2O | | 0 | 0 | 0 | | 1 | | 0 |
| CH4 | | 0 | 0 | 0 | | 0 | | 0 |
| O2 | | 0.21 | 0 | 0 | | 0 | | 0 |
| N2 | | 0.79 | 0 | 0 | | 0 | | 0 |
| CO2 | | 0 | 0 | 0 | | 0 | | 0 |
| Mass Flows | kg/hr | 645702.4059 | 293190.91 | 293190.91 | | 87957.27 | | 8659557.214 |
| C2H6 | kg/hr | 0 | 293190.91 | 293190.91 | | 0 | | 0 |
| C2H4 | kg/hr | 0 | 0 | 0 | | 0 | | 0 |
| H2 | kg/hr | 0 | 0 | 0 | | 0 | | 0 |
| HE | kg/hr | 0 | 0 | 0 | | 0 | | 8659557.214 |
| H2O | kg/hr | 0 | 0 | 0 | | 87957.27 | | 0 |
| CH4 | kg/hr | 0 | 0 | 0 | | 0 | | 0 |
| O2 | kg/hr | 150395.0681 | 0 | 0 | | 0 | | 0 |
| N2 | kg/hr | 495307.3378 | 0 | 0 | | 0 | | 0 |
| CO2 | kg/hr | 0 | 0 | 0 | | 0 | | 0 |
| Mass Fractions | | | | | | | | |
| C2H6 | | 0 | 1 | 1 | | 0 | | 0 |
| C2H4 | | 0 | 0 | 0 | | 0 | | 0 |
| H2 | | 0 | 0 | 0 | | 0 | | 0 |
| HE | | 0 | 0 | 0 | | 0 | | 1 |
| H2O | | 0 | 0 | 0 | | 1 | | 0 |
| CH4 | | 0 | 0 | 0 | | 0 | | 0 |
| O2 | | 0.232917001 | 0 | 0 | | 0 | | 0 |
| N2 | | 0.767082999 | 0 | 0 | | 0 | | 0 |
| CO2 | | 0 | 0 | 0 | | 0 | | 0 |
| Volume Flow | | 76.08932828 | 9.660288211 | 26.11274563 | | 6.029460462 | | 860.1837133 |
| Liquid Phase | | | | | | | | |
| Molar Enthalpy | | | | | −285689787.5 | | −285702340.6 | |
| Mass Enthalpy | | | | | −15858193.02 | | −15858889.82 | |
| Molar Entropy | | | | | −161414.8636 | | −161413.1772 | |
| Mass Entropy | | | | | −8959.886472 | | −8959.792867 | |
| Molar Density | | | | | 53.81392819 | | 53.81563816 | |
| Mass Density | | | | | 969.4729842 | | 969.5037899 | |
| Enthalpy Flow | | | | | −387.4564902 | | −387.473515 | |
| Average MW | | | | | 18.01528 | | 18.01528 | |
| Mole Flows | kmol/hr | | | | 4882.37041 | | 4882.37041 | |
| C2H6 | kmol/hr | | | | 0 | | 0 | |
| C2H4 | kmol/hr | | | | 0 | | 0 | |
| H2 | kmol/hr | | | | 0 | | 0 | |
| HE | kmol/hr | | | | 0 | | 0 | |
| H2O | kmol/hr | | | | 4882.37041 | | 4882.37041 | |
| CH4 | kmol/hr | | | | 0 | | 0 | |
| O2 | kmol/hr | | | | 0 | | 0 | |
| N2 | kmol/hr | | | | 0 | | 0 | |
| CO2 | kmol/hr | | | | 0 | | 0 | |
| Mole Fractions | | | | | | | | |
| C2H6 | | | | | 0 | | 0 | |
| C2H4 | | | | | 0 | | 0 | |
| H2 | | | | | 0 | | 0 | |
| HE | | | | | 0 | | 0 | |
| H2O | | | | | 1 | | 1 | |
| CH4 | | | | | 0 | | 0 | |
| O2 | | | | | 0 | | 0 | |
| N2 | | | | | 0 | | 0 | |
| CO2 | | | | | 0 | | 0 | |
| Mass Flows | kg/hr | | | | 87957.27 | | 87957.27 | |
| C2H6 | kg/hr | | | | 0 | | 0 | |
| C2H4 | kg/hr | | | | 0 | | 0 | |
| H2 | kg/hr | | | | 0 | | 0 | |
| HE | kg/hr | | | | 0 | | 0 | |
| H2O | kg/hr | | | | 87957.27 | | 87957.27 | |
| CH4 | kg/hr | | | | 0 | | 0 | |
| O2 | kg/hr | | | | 0 | | 0 | |
| N2 | kg/hr | | | | 0 | | 0 | |
| CO2 | kg/hr | | | | 0 | | 0 | |
| Mass Fractions | | | | | | | | |
| C2H6 | | | | | 0 | | 0 | |
| C2H4 | | | | | 0 | | 0 | |
| H2 | | | | | 0 | | 0 | |
| HE | | | | | 0 | | 0 | |
| H2O | | | | | 1 | | 1 | |
| CH4 | | | | | 0 | | 0 | |
| O2 | | | | | 0 | | 0 | |
| N2 | | | | | 0 | | 0 | |
| CO2 | | | | | 0 | | 0 | |
| Volume Flow | | | | | 0.025201914 | | 0.025201113 | |

| | | FEED | FLUE | FUEL | GAS-P | LIQ-P | P-HE-1 | PRODUCT |
|---|---|---|---|---|---|---|---|---|
| | | From | | | | | | |
| | | MIXER | FURNACE | | SCRUBBER | SCRUBBER | HE-HX | REACTOR |
| | | To | | | | | | |
| | | REACTOR | | FURNACE | | | | SCRUBBER |
| | | Stream Class | | | | | | |
| Description | Units | CONVEN | CONVEN | CONVEN | CONVEN | CONVEN | CONVEN | CONVEN |
| Maximum | | | | | | | | |
| Relative Error | | | | | | | | |
| Cost Flow | $/sec | | | | | | | |
| MIXED | | | | | | | | |
| Substream | | | | | | | | |
| Phase | | Vapor Phase | Vapor Phase | Vapor Phase | Vapor Phase | Liquid Phase | Vapor Phase | Vapor Phase |
| Temperature | C | 626.9486183 | 140 | 25 | 50 | 50 | 710 | 700 |
| Pressure | kPag | 695 | 100 | 101 | 97 | 97 | 5889 | 197 |
| Molar Vapor | | 1 | 1 | 1 | 1 | 0 | 1 | 1 |
| Fraction | | | | | | | | |
| Molar Liquid | | 0 | 0 | 0 | 0 | 1 | 0 | 0 |
| Fraction | | | | | | | | |
| Molar Solid | | 0 | 0 | 0 | 0 | 0 | 0 | 0 |
| Fraction | | | | | | | | |
| Mass Vapor | | 1 | 1 | 1 | 1 | 0 | 1 | 1 |
| Fraction | | | | | | | | |
| Mass Liquid | | 0 | 0 | 0 | 0 | 1 | 0 | 0 |
| Fraction | | | | | | | | |
| Mass Solid | | 0 | 0 | 0 | 0 | 0 | 0 | 0 |
| Fraction | | | | | | | | |
| Molar Enthalpy | J/kmol | −94283020.59 | −73482852.3 | −14970559.09 | −9551856.59 | −285701221.6 | 14315440.34 | −17692763.71 |
| Mass Enthalpy | J/kg | −3619645.923 | −2799644.962 | −3097332.469 | −524464.8984 | −15858802.05 | 3576535.338 | −973444.3297 |
| Molar Entropy | J/kmol-K | −69584.64382 | −26.31544403 | −17781.08828 | −49573.14932 | −161412.5666 | −9121.984442 | 7909.517884 |
| Mass Entropy | J/kg-K | −2671.443604 | −1.002599899 | −3678.816651 | −2721.918663 | −8959.74448 | −2279.014751 | 435.1765198 |
| Molar Density | kmol/cum | 0.106468778 | 0.058710989 | 0.081564659 | 0.074137483 | 53.81531384 | 0.725608372 | 0.036852349 |
| Mass Density | kg/cum | 2.773254124 | 1.5409993 | 0.394232312 | 1.350234512 | 969.4995154 | 2.90432007 | 0.669807087 |
| Enthalpy Flow | MW | −383.2281821 | −522.1924117 | −22.17430242 | −45.16206831 | −313.4292336 | 8603.114552 | −103.0629263 |
| Average MW | | 26.04758106 | 26.24720395 | 4.833371697 | 18.21257556 | 18.01530914 | 4.0026 | 18.17542428 |
| Mole Flows | kmol/hr | 14632.76682 | 25582.73969 | 5332.298427 | 17021.13556 | 3949.388928 | 2163483.04 | 20970.52449 |
| C2H6 | kmol/hr | 9750.396413 | 0 | 0 | 3412.635292 | 0.003452927 | 0 | 3412.638745 |
| C2H4 | kmol/hr | 0 | 0 | 0 | 6337.748652 | 0.009016704 | 0 | 6337.757669 |
| H2 | kmol/hr | 0 | 0 | 4261.232976 | 6337.756603 | 0.001065232 | 0 | 6337.757669 |
| HE | kmol/hr | 0 | 0 | 0 | 0 | 0 | 2163483.04 | 0 |
| H2O | kmol/hr | 4882.37041 | 6403.363877 | 0 | 932.9950173 | 3949.375393 | 0 | 4882.37041 |
| CH4 | kmol/hr | 0 | 0 | 1071.06545 | 0 | 0 | 0 | 0 |
| O2 | kmol/hr | 0 | 427.2747389 | 0 | 0 | 0 | 0 | 0 |
| N2 | kmol/hr | 0 | 17681.03562 | 0 | 0 | 0 | 0 | 0 |
| CO2 | kmol/hr | 0 | 1071.06545 | 0 | 0 | 0 | 0 | 0 |
| Mole Fractions | | | | | | | | |
| C2H6 | | 0.666339902 | 0 | 0 | 0.200493985 | 8.74E−07 | 0 | 0.162735021 |
| C2H4 | | 0 | 0 | 0 | 0.372345818 | 2.28E−06 | 0 | 0.302222182 |
| H2 | | 0 | 0 | 0.799136251 | 0.372346286 | 2.70E−07 | 0 | 0.302222182 |
| HE | | 0 | 0 | 0 | 0 | 0 | 1 | 0 |
| H2O | | 0.333660098 | 0.250300162 | 0 | 0.054813911 | 0.999996573 | 0 | 0.232820615 |
| CH4 | | 0 | 0 | 0.200863749 | 0 | 0 | 0 | 0 |
| O2 | | 0 | 0.01670168 | 0 | 0 | 0 | 0 | 0 |
| N2 | | 0 | 0.691131436 | 0 | 0 | 0 | 0 | 0 |
| CO2 | | 0 | 0.041866722 | 0 | 0 | 0 | 0 | 0 |
| Mass Flows | kg/hr | 381148.18 | 671475.3862 | 25772.9803 | 309998.7175 | 71149.46245 | 8659557.214 | 381148.18 |
| C2H6 | kg/hr | 293190.91 | 0 | 0 | 102616.7147 | 0.103828282 | 0 | 102616.8185 |
| C2H4 | kg/hr | 0 | 0 | 0 | 177797.6796 | 0.252952442 | 0 | 177797.9326 |
| H2 | kg/hr | 0 | 0 | 8590.134333 | 12776.15678 | 0.00214738 | 0 | 12776.15893 |
| HE | kg/hr | 0 | 0 | 0 | 0 | 0 | 8659557.214 | 0 |
| H2O | kg/hr | 87957.27 | 115358.3932 | 0 | 16808.16648 | 71149.10352 | 0 | 87957.27 |
| CH4 | kg/hr | 0 | 0 | 17182.84596 | 0 | 0 | 0 | 0 |
| O2 | kg/hr | 0 | 13672.27891 | 0 | 0 | 0 | 0 | 0 |
| N2 | kg/hr | 0 | 495307.3378 | 0 | 0 | 0 | 0 | 0 |
| CO2 | kg/hr | 0 | 47137.37625 | 0 | 0 | 0 | 0 | 0 |
| Mass Fractions | | | | | | | | |
| C2H6 | | 0.769230775 | 0 | 0 | 0.33102303 | 1.46E−06 | 0 | 0.269230771 |
| C2H4 | | 0 | 0 | 0 | 0.573543275 | 3.56E−06 | 0 | 0.466479815 |
| H2 | | 0 | 0 | 0.3333 | 0.041213579 | 3.02E−08 | 0 | 0.033520189 |
| HE | | 0 | 0 | 0 | 0 | 0 | 1 | 0 |
| H2O | | 0.230769225 | 0.171798394 | 0 | 0.054220116 | 0.999994955 | 0 | 0.230769225 |
| CH4 | | 0 | 0 | 0.6667 | 0 | 0 | 0 | 0 |
| O2 | | 0 | 0.020361549 | 0 | 0 | 0 | 0 | 0 |
| N2 | | 0 | 0.737640348 | 0 | 0 | 0 | 0 | 0 |
| CO2 | | 0 | 0.070199708 | 0 | 0 | 0 | 0 | 0 |
| Volume Flow | cum/sec | 38.17698981 | 121.0389522 | 18.15975243 | 63.7746659 | 0.020385507 | 828.2257125 | 158.0671457 |
| Vapor Phase | | | | | | | | |
| Molar Enthalpy | | −94283021.05 | −73482852.3 | −14970559.09 | −9551856.59 | | 14315440.34 | −17692763.71 |
| Mass Enthalpy | | −3619645.94 | −2799644.962 | −3097332.469 | −524464.8984 | | 3576535.338 | −973444.3297 |
| Molar Entropy | | −69584.64382 | −26.31544403 | −17781.08828 | −49573.14932 | | −9121.984442 | 7909.517884 |
| Mass Entropy | | −2671.443604 | −1.002599899 | −3678.816651 | −2721.918663 | | −2279.014751 | 435.1765198 |
| Molar Density | | 0.106468778 | 0.058710989 | 0.081564659 | 0.074137483 | | 0.725608372 | 0.036852349 |
| Mass Density | | 2.773254124 | 1.5409993 | 0.394232312 | 1.350234512 | | 2.90432007 | 0.669807087 |
| Enthalpy Flow | | −383.228184 | −522.1924117 | −22.17430242 | −45.16206831 | | 8603.114552 | −103.0629263 |
| Average MW | | 26.04758106 | 26.24720395 | 4.833371697 | 18.21257556 | | 4.0026 | 18.17542428 |
| Mole Flows | kmol/hr | 14632.76682 | 25582.73969 | 5332.298427 | 17021.13556 | | 2163483.04 | 20970.52449 |
| C2H6 | kmol/hr | 9750.396413 | 0 | 0 | 3412.635292 | | 0 | 3412.638745 |
| C2H4 | kmol/hr | 0 | 0 | 0 | 6337.748652 | | 0 | 6337.757669 |
| H2 | kmol/hr | 0 | 0 | 4261.232976 | 6337.756603 | | 0 | 6337.757669 |
| HE | kmol/hr | 0 | 0 | 0 | 0 | | 2163483.04 | 0 |
| H2O | kmol/hr | 4882.37041 | 6403.363877 | 0 | 932.9950173 | | 0 | 4882.37041 |
| CH4 | kmol/hr | 0 | 0 | 1071.06545 | 0 | | 0 | 0 |
| O2 | kmol/hr | 0 | 427.2747389 | 0 | 0 | | 0 | 0 |
| N2 | kmol/hr | 0 | 17681.03562 | 0 | 0 | | 0 | 0 |
| CO2 | kmol/hr | 0 | 1071.06545 | 0 | 0 | | 0 | 0 |
| Mole Fractions | | | | | | | | |
| C2H6 | | 0.666339902 | 0 | 0 | 0.200493985 | | 0 | 0.162735021 |
| C2H4 | | 0 | 0 | 0 | 0.372345818 | | 0 | 0.302222182 |
| H2 | | 0 | 0 | 0.799136251 | 0.372346286 | | 0 | 0.302222182 |
| HE | | 0 | 0 | 0 | 0 | | 1 | 0 |
| H2O | | 0.333660098 | 0.250300162 | 0 | 0.054813911 | | 0 | 0.232820615 |
| CH4 | | 0 | 0 | 0.200863749 | 0 | | 0 | 0 |
| O2 | | 0 | 0.01670168 | 0 | 0 | | 0 | 0 |
| N2 | | 0 | 0.691131436 | 0 | 0 | | 0 | 0 |
| CO2 | | 0 | 0.041866722 | 0 | 0 | | 0 | 0 |
| Mass Flows | kg/hr | 381148.18 | 671475.3862 | 25772.9803 | 309998.7175 | | 8659557.214 | 381148.18 |
| C2H6 | kg/hr | 293190.91 | 0 | 0 | 102616.7147 | | 0 | 102616.8185 |
| C2H4 | kg/hr | 0 | 0 | 0 | 177797.6796 | | 0 | 177797.9326 |
| H2 | kg/hr | 0 | 0 | 8590.134333 | 12776.15678 | | 0 | 12776.15893 |
| HE | kg/hr | 0 | 0 | 0 | 0 | | 8659557.214 | 0 |
| H2O | kg/hr | 87957.27 | 115358.3932 | 0 | 16808.16648 | | 0 | 87957.27 |
| CH4 | kg/hr | 0 | 0 | 17182.84596 | 0 | | 0 | 0 |
| O2 | kg/hr | 0 | 13672.27891 | 0 | 0 | | 0 | 0 |
| N2 | kg/hr | 0 | 495307.3378 | 0 | 0 | | 0 | 0 |
| CO2 | kg/hr | 0 | 47137.37625 | 0 | 0 | | 0 | 0 |
| Mass Fractions | | | | | | | | |
| C2H6 | | 0.769230775 | 0 | 0 | 0.33102303 | | 0 | 0.269230771 |
| C2H4 | | 0 | 0 | 0 | 0.573543275 | | 0 | 0.466479815 |
| H2 | | 0 | 0 | 0.3333 | 0.041213579 | | 0 | 0.033520189 |
| HE | | 0 | 0 | 0 | 0 | | 1 | 0 |
| H2O | | 0.230769225 | 0.171798394 | 0 | 0.054220116 | | 0 | 0.230769225 |
| CH4 | | 0 | 0 | 0.6667 | 0 | | 0 | 0 |
| O2 | | 0 | 0.020361549 | 0 | 0 | | 0 | 0 |
| N2 | | 0 | 0.737640348 | 0 | 0 | | 0 | 0 |
| CO2 | | 0 | 0.070199708 | 0 | 0 | | 0 | 0 |
| Volume Flow | | 38.17698981 | 121.0389522 | 18.15975243 | 63.7746659 | | 828.2257125 | 158.0671457 |
| Liquid Phase | | | | | | | | |
| Molar Enthalpy | | | | | | −285701221.6 | | |
| Mass Enthalpy | | | | | | −15858802.05 | | |
| Molar Entropy | | | | | | −161412.5666 | | |
| Mass Entropy | | | | | | −8959.74448 | | |
| Molar Density | | | | | | 53.81531384 | | |
| Mass Density | | | | | | 969.4995154 | | |
| Enthalpy Flow | | | | | | −313.4292336 | | |
| Average MW | | | | | | 18.01530914 | | |
| Mole Flows | kmol/hr | | | | | 3949.388928 | | |
| C2H6 | kmol/hr | | | | | 0.003452927 | | |
| C2H4 | kmol/hr | | | | | 0.009016704 | | |
| H2 | kmol/hr | | | | | 0.001065232 | | |
| HE | kmol/hr | | | | | 0 | | |
| H2O | kmol/hr | | | | | 3949.375393 | | |
| CH4 | kmol/hr | | | | | 0 | | |
| O2 | kmol/hr | | | | | 0 | | |
| N2 | kmol/hr | | | | | 0 | | |
| CO2 | kmol/hr | | | | | 0 | | |
| Mole Fractions | | | | | | | | |
| C2H6 | | | | | | 8.74E−07 | | |
| C2H4 | | | | | | 2.28E−06 | | |
| H2 | | | | | | 2.70E−07 | | |
| HE | | | | | | 0 | | |
| H2O | | | | | | 0.999996573 | | |
| CH4 | | | | | | 0 | | |
| O2 | | | | | | 0 | | |
| N2 | | | | | | 0 | | |
| CO2 | | | | | | 0 | | |
| Mass Flows | kg/hr | | | | | 71149.46245 | | |
| C2H6 | kg/hr | | | | | 0.103828282 | | |
| C2H4 | kg/hr | | | | | 0.252952442 | | |
| H2 | kg/hr | | | | | 0.00214738 | | |
| HE | kg/hr | | | | | 0 | | |
| H2O | kg/hr | | | | | 71149.10352 | | |
| CH4 | kg/hr | | | | | 0 | | |
| O2 | kg/hr | | | | | 0 | | |
| N2 | kg/hr | | | | | 0 | | |
| CO2 | kg/hr | | | | | 0 | | |
| Mass Fractions | | | | | | | | |
| C2H6 | | | | | | 1.46E−06 | | |
| C2H4 | | | | | | 3.56E−06 | | |
| H2 | | | | | | 3.02E−08 | | |
| HE | | | | | | 0 | | |
| H2O | | | | | | 0.999994955 | | |
| CH4 | | | | | | 0 | | |
| O2 | | | | | | 0 | | |
| N2 | | | | | | 0 | | |
| CO2 | | | | | | 0 | | |
| Volume Flow | | | | | | 0.020385507 | | |

1. A steam cracking system, comprising:
a small modular nuclear reactor (SMR) to provide a heat exchange stream;
a steam heat exchanger to use the heat exchange stream to generate a steam stream from a water stream, wherein the steam stream is provided to a steam-cracking furnace;
a feed heat exchanger to use the heat exchange stream to generate a hot feed stream, wherein the hot feed stream is provided to the steam-cracking furnace; and
the steam-cracking furnace to generate a product stream comprising an olefin.

2. The steam cracking system of claim 1, wherein the steam-cracking furnace comprises an internal heat exchanger coupled to the heat exchange stream.

3. The steam cracking system of claim 1, wherein the steam-cracking furnace comprises an internal heat exchanger coupled to a fuel gas stream, wherein the internal heat exchanger is configured to combust the fuel gas stream.

4. The steam cracking system of claim 3, comprising a fuel gas heat exchanger to heat the fuel gas stream using the heat exchange stream.

5. The steam cracking system of claim 3, wherein the internal heat exchanger is coupled to an oxidizer stream, wherein the oxidizer stream is heated in an oxidizer heat exchanger.

6. The steam cracking system of claim 1, wherein the heat exchange stream comprises a gas stream heated in the SMR.

7. The steam cracking system of claim 6, wherein the gas stream comprises helium.

8. The steam cracking system of claim 6, wherein the gas stream comprises carbon dioxide.

9. The steam cracking system of claim 1, comprising a nuclear coolant heat exchanger to generate the heat exchange stream.

10. The steam cracking system of claim 9, wherein the nuclear coolant comprises a liquid metal.

11. The steam cracking system of claim 9, wherein the nuclear coolant comprises a molten salt.

12. The steam cracking system of claim 9, wherein the nuclear coolant comprises a hot gas.

13-21. (canceled)

22. The steam cracking system of claim 1, comprising an electrolytic water splitter, wherein the water splitter generates oxygen and hydrogen.

23. The steam cracking system of claim 22, wherein the electrolytic water splitter is powered by electricity generated in the steam cracking system.

24. The steam cracking system of claim 22, wherein the electrolytic water splitter is fed a boiler feed water.

25. The steam cracking system of claim 1, comprising a thermochemical water splitter, wherein the thermochemical water splitter is coupled to the heat exchange stream, wherein the thermochemical water splitter is fed a low-pressure steam wherein the water splitter generates oxygen and hydrogen.

26. The steam cracking system of claim 25, wherein the thermochemical water splitter uses an iron catalytic cycle.

27. The steam cracking system of claim 26, wherein the thermochemical water splitter is coupled to the heat exchange stream, and wherein the water splitter is fed a low-pressure steam.

28. The steam cracking system of claim 25, wherein the thermochemical water splitter uses a copper catalytic cycle, and wherein the copper catalytic cycle comprises an electrolytic reaction.

29. The steam cracking system of claim 28, wherein the thermochemical water splitter is powered by electricity generated by the turbine in the steam cracking system.

30. A method for generating heat for a steam cracking process, comprising:
heating a coolant stream in a small modular nuclear reactor (SMR) to create a high-temperature coolant stream;
generating a high-pressure steam stream from the high-temperature coolant stream, creating a first portion of a low-temperature coolant stream;
providing the high-pressure steam stream to a steam-cracking furnace;
generating a hot feed stream from the high-temperature coolant stream, creating a second portion of the low-temperature coolant stream;
providing the hot feed stream to the steam-cracking furnace; and
generating a product stream comprising an alpha olefin from the steam-cracking furnace.

31-44. (canceled)

45. A steam cracking process, comprising:
heating a coolant stream in a small modular nuclear reactor (SMR) to create a high-temperature coolant stream;
generating a high-pressure steam stream from the high-temperature coolant stream, creating a first portion of a low-temperature coolant stream;
providing the high-pressure steam stream to a steam-cracking furnace;
generating a hot feed stream from the high-temperature coolant stream, creating a second portion of the low-temperature coolant stream, wherein the hot feed stream comprises a C₂ to C₆ alkane;
providing the hot feed stream to the steam-cracking furnace; and
generating a product stream comprising a C₂ to C₆ alpha olefin from the steam-cracking furnace.

46-49. (canceled)
